(19) United States
(12) Patent Application Publication
(10) Pub. No.: US 2024/0254162 A1
(43) Pub. Date: Aug. 1, 2024

(54) CYCLOTHEONELLAZOLEA, AND SYNTHETIC METHOD THEREFOR, AND APPLICATION METHOD THEREOF

(71) Applicant: The Second People's Hospital of Shenzhen(Shenzhen Institute of Geriatrics), Shenzhen (CN)

(72) Inventors: Zhengzhi Wu, Shenzhen (CN); Bohua Long, Shenzhen (CN); Liuyang Pu, Shenzhen (CN); Zhiyue Li, Shenzhen (CN); Shengquan Hu, Shenzhen (CN); Limin Li, Shenzhen (CN)

(21) Appl. No.: 18/527,402

(22) Filed: Dec. 4, 2023

(30) Foreign Application Priority Data
Dec. 5, 2022 (CN) 2022115515895

(51) Int. Cl. C07K 5/02; C07C 227/20; C07K 1/107; C07K 5/062

(52) U.S. Cl. CPC C07K 5/0202; C07C 227/20; C07K 1/107; C07K 5/06026; A61K 38/00

(57) ABSTRACT

A CyclotheonellazoleA, a synthesis method therefor, and an application method thereof are provided, and a compound with a structure of formula (I) and a pharmaceutically acceptable salt thereof are provided, the structure of formula (I) is as follows:

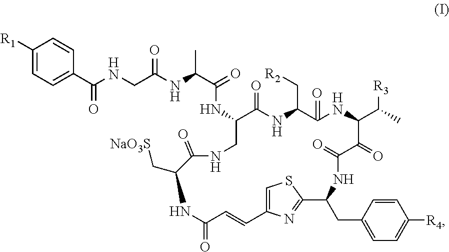

R₁, R₂, R₃ and R₄ are independently selected form the group consisting of a hydrogen group (H), an alkyl group with carbon atoms in a range of 1-6, an alkoxy group with carbon atoms in a range of 1-6, a halogen group, a hydroxyl group, an amino group, a nitro group, a cyano group and a sulfydryl group. A CyclotheonellazoleA natural product is successfully synthesized by a classical retrosynthesis analysis, a stereoscopic structure of the CyclotheonellazoleA natural product is confirmed, extremely excellent protease inhibitory activity of the CyclotheonellazoleA natural product is confirmed by combining a protease inhibition activity experiment, and the CyclotheonellazoleA has a strong application prospect in a pharmaceutical industry.

### TECHNICAL FIELD

The disclosure relates to the technical field of drugs, and more particularly to a CyclotheonellazoleA, and a synthetic method therefor, and an application method thereof.

### BACKGROUND

Vaccines are considered the most effective method to block severe acute respiratory syndrome coronavirus 2 (SARS-COV-2) virus at present. However, a sensitivity of virus to vaccine-induced immunity is decreased with an emergence of Corona Virus Disease 2019 (Covid-19) strain, therefore, developing drugs for SARS-COV-2 virus is a top priority at present.

A specific path of the SARS-COV-2 virus infecting host cells is as follows: the SARS-CoV-2 virus are combined with receptor protein angiotensin converting enzyme 2 in the host cells, the SARS-COV-2 virus are fused with host cell membrane under an action of transmembrane serine protease 2 (TMPRSS2), viral genome is released into cytoplasm of the host cells, and a transcriptional replication is further performed under an action of mainprotease (Mpro) of the SARS-COV-2 virus to complete an assembly of progeny virus, to thereby release the progeny virus to outsides of the host cells.

Neutrophil elastase (NE) is a serine proteinase widely expressed in pancreas and neutrophils. Under a normal physiological condition, the NE can clear bacteria and damaged tissues, and promote tissue regeneration, however, under a pathological condition, overexpression of the NE can damage blood vessels, thus leading to inflammation, viral or bacterial infections. Research has shown that, in a COVID-19 patient, the NE released by the neutrophils can activate epithelial sodium ion (Na⁺) transporters, leading to hypertension and pulmonary airway dehydration, thus resulting in reduced mucociliary clearance efficiency, lung barrier dysfunction and pro-inflammatory cytokine release, and ultimately leading to severe acute lung injury/acute respiratory distress syndrome (ALI/ARDS) in the COVID-19 patient. Therefore, the TMPRSS2, the Mpro and the NE are all considered as potential targets for a treatment of the SARS-COV-2.

A CyclotheonellazoleA (CA) is a natural product of macrocyclic polypeptide isolated from sponge by Carmeli et al. of Tel Aviv University, Israel. Structurally, the CA is composed of 8 amino acids, 6 of which are non-proteinogenic amino acids and contain 7 chiral centers, and the structure of the CA is as follows:

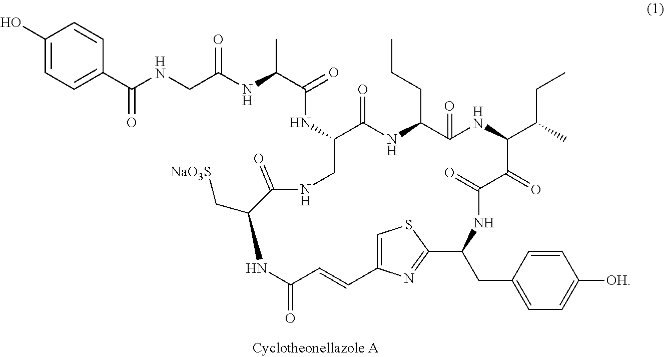

It attracts more interest of people to further discover a wider application of the CA in protease inhibition based on research results of partial protease inhibitory activity of the CA. In addition, due to a complexity of the structure of the CA, it has numerous stereoisomers, and biological activities between different stereoisomers often varies greatly. Research has found more natural stereoisomer products and confirmed their protease inhibitory activity, which is of great value for research of structure-activity relationships and reserve of candidate drugs for enzyme inhibitors.

### SUMMARY

In order to solve problems in the related art, embodiments of the disclosure provide a CyclotheonellazoleA, and a synthetic method therefor, and an application method thereof. Technical solutions are as follows.

According to a first aspect, a compound or a pharmaceutically acceptable salt thereof are provided, and the compound has a structure represented by a general formula (I) expressed as follows:

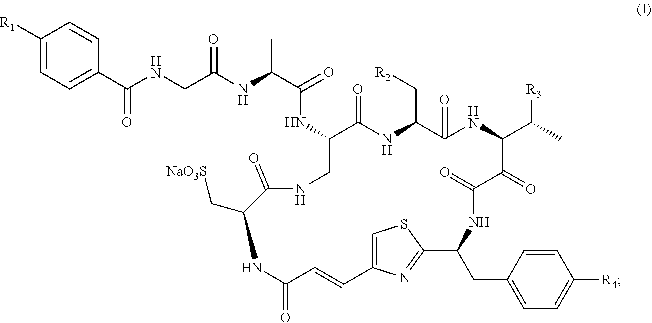

where R₁, R₂, R₃ and R₄ are independently selected form the group consisting of a hydrogen group (H), an alkyl group with a carbon atom in a range of 1-6, an alkoxy group with a carbon atom in a range of 1-6, a halogen group, a hydroxyl group, an amino group, a nitro group, a cyano group and a sulfydryl group.

In an embodiment, the R₁ is the hydroxyl group, the R₂ is the alkyl group with the carbon atom in a range of 1-3, the R₃ is the alkyl group with the carbon atom in a range of 1-3, and the R₄ is the hydroxyl group.

In an embodiment, the compound has a structure represented by a formula (la) expressed as follows:

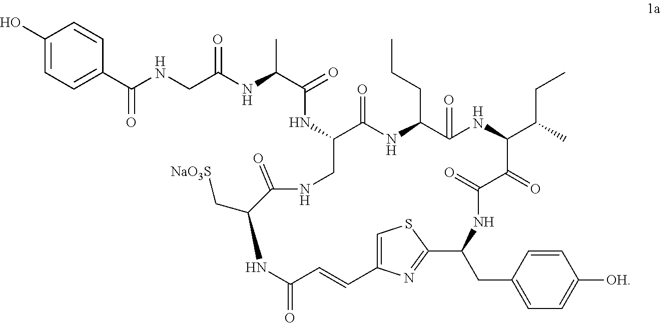

According to a second aspect, a synthetic method of the CyclotheonellazoleA is provided, and the method includes:
removing a 9-fluorenylmethyloxycarbonyl (Fmoc) protection group from a compound 2 with diethylamine (DEA) to obtain a first group-removed compound, and performing a peptide grafting reaction on the first group-removed compound and a compound 30 to obtain a compound 31; and removing a t-butyloxy carbonyl (Boc) protection group from the compound 31 with trifluoroacetic acid (TFA) to obtain a second group-removed compound, and performing a peptide grafting reaction on the second group-removed compound and a compound 32 to obtain a compound 33;
where a specific synthesis process of the compound 33 is as follows:

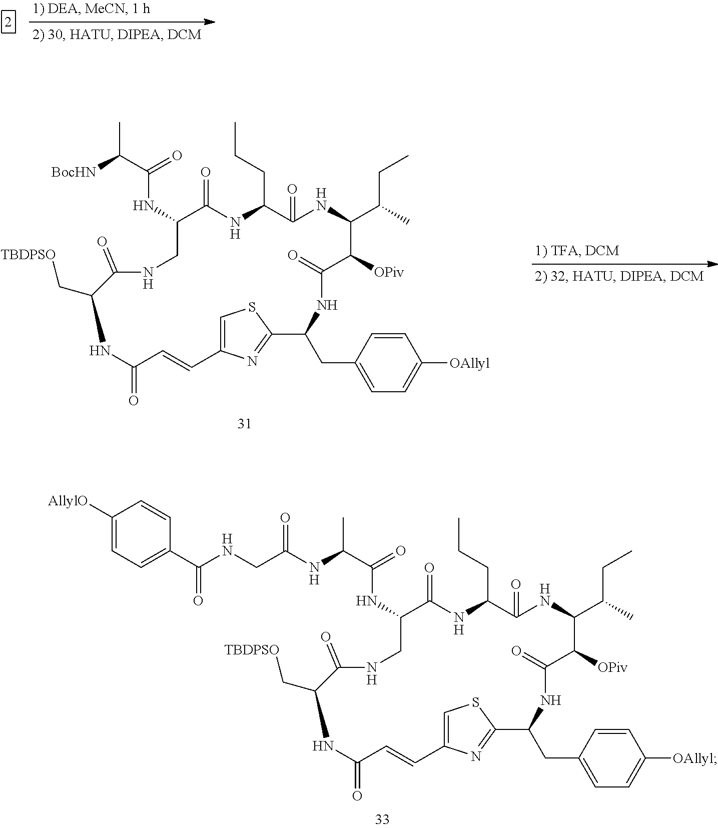

removing a tert-butyldiphenylsilyl (TBDPS) protection group from the compound 33 with ammonium fluoride (NH₄F) to obtain a third group-removed compound, and activating a hydroxyl group of the third group-removed compound with methanesulfonyl chloride (MsCl) to obtain activated compound; performing a bimolecular nucleophilic substitution (SN₂) reaction on the activated compound with potassium thioacetate (KSAc) to obtain a thioester; oxidizing the thioester with potassium monopersulphate triple salt (Oxone) to obtain a first sulfurous acid; removing a pivaloyl (Piv) group from the first sulfurous acid with triethylamine (C₆H₁₅N) in a reflux condition of methanol (MeOH) to obtain a secondary alcohol; oxidizing the secondary alcohol with 2-iodoxybenzoic acid (IBX) to obtain a α-ketoamide; and oxidizing the α-ketoamide with selenium dioxide (SeO₂) to remove two allyl groups from the α-ketoamide, to thereby obtain a second sulfurous acid, and adding a saturated sodium chloride solution into the second sulfurous acid to obtain the CyclotheonellazoleA;
where a specific synthesis process of the CyclotheonellazoleA is as follows:

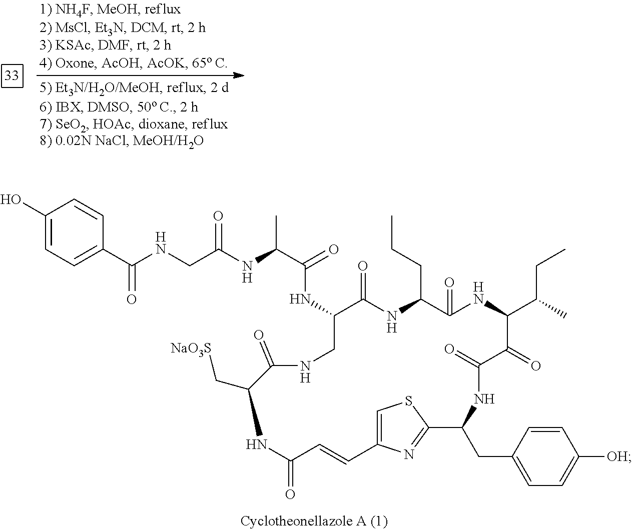

where structures of the compound 2, the compound 30 and the compound 33 are as follows:

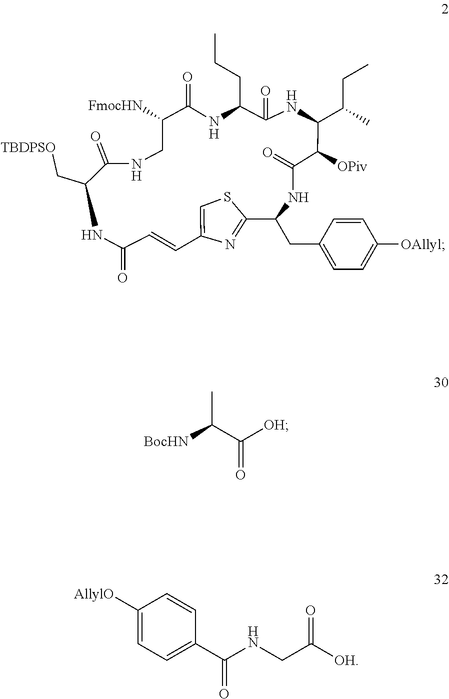

In an embodiment, a synthetic method of the compound 2 includes:
performing a condensation reaction on a compound 19 and a compound 20 to obtain a compound 21, protecting a primary alcohol group of the compound 21 with tert-butyldimethylsilyl chloride (TBSCL) to obtain a compound 22, and performing a thionation reaction on the compound 22 with a Lawesson's reagent to obtain a compound 23; removing a t-butyldimethylsilyl (TBS) protection group from the compound 23 with the NH₄F to obtain a compound 24, performing a ring-closing reaction on the compound 24 with diethylaminosulfurtrifluoride (DAST) to obtain a ring-closed compound, oxidizing the ring-closed compound with 1,8-diazabicyclo[5.4.0]undec-7-ene (DBU) and bromotrichloro methane (CBrCl₃) to obtain a first oxidized compound, and adding an allyl group into the first oxidized compound to obtain a compound 25; reducing a methyl ester group of the compound 25 with sodium borohydride (NaBH₄) to obtain a reduced compound, oxidizing a primary alcohol group of the reduced compound with the IBX to obtain a second oxidized compound, and performing a Wittig reaction on the second oxidized compound to obtain a compound 26; and hydrolyzing an ethyl ester group of the compound 26 with sodium hydroxide (NaOH) to obtain a first hydrolyzed compound, and performing an esterification reaction on the first hydrolyzed compound and trichloroethanol (C₂H₃Cl₃O) to obtain a compound 27;
where a specific synthesis process of the compound 27 is as follows:

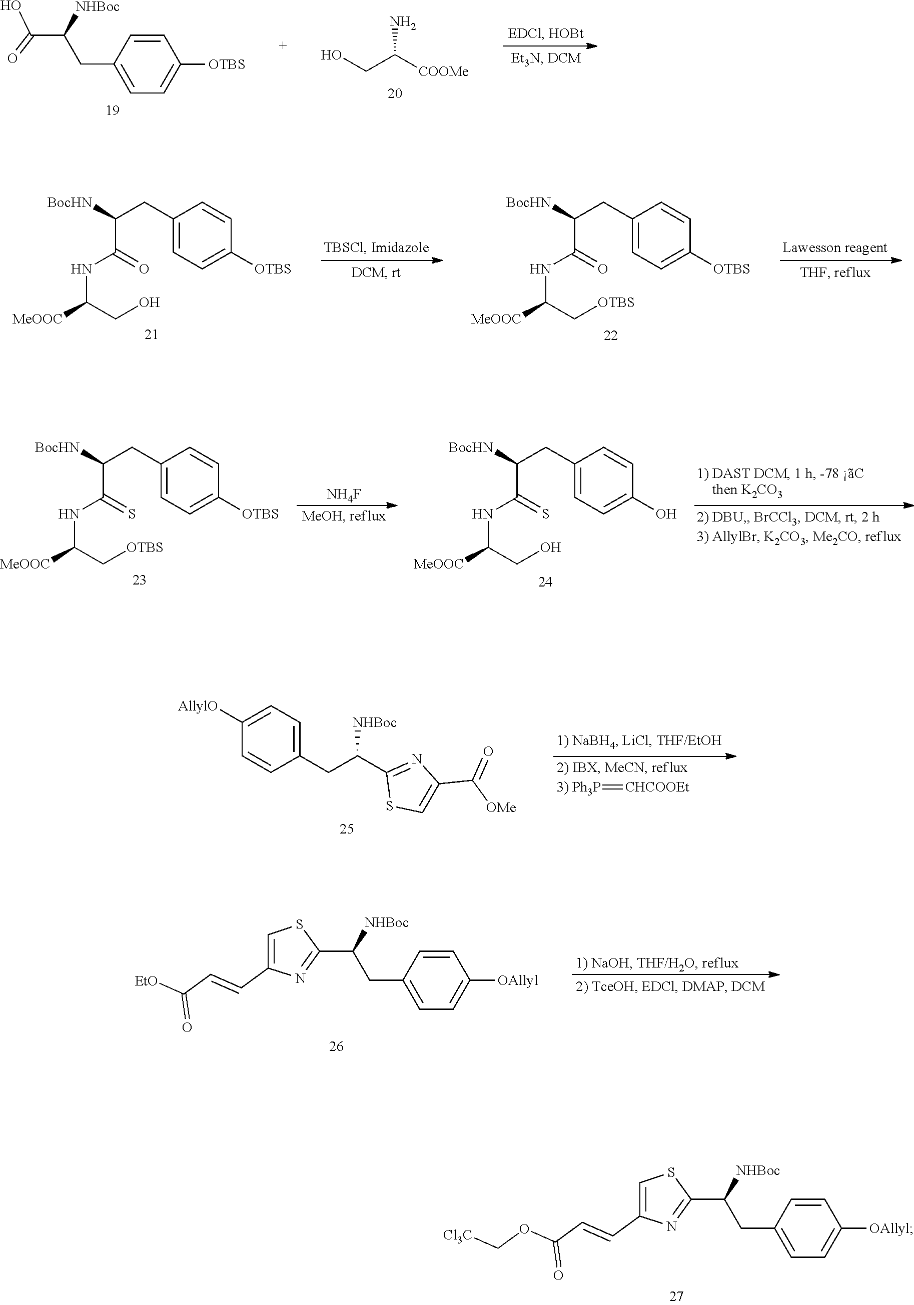

removing a Boc protection group from the compound 27 with the trifluoroacetic acid to obtain a fourth group-removed compound, and performing a peptide grafting reaction on the fourth group-removed compound and a compound 18 to obtain a compound 28; and removing a Boc protection group from the compound 28 with the trifluoroacetic acid to obtain a fifth group-removed compound, and performing a peptide grafting reaction on the fifth group-removed compound and a compound 11 to obtain a compound 29;
where a specific synthesis process of the compound 29 is as follows:

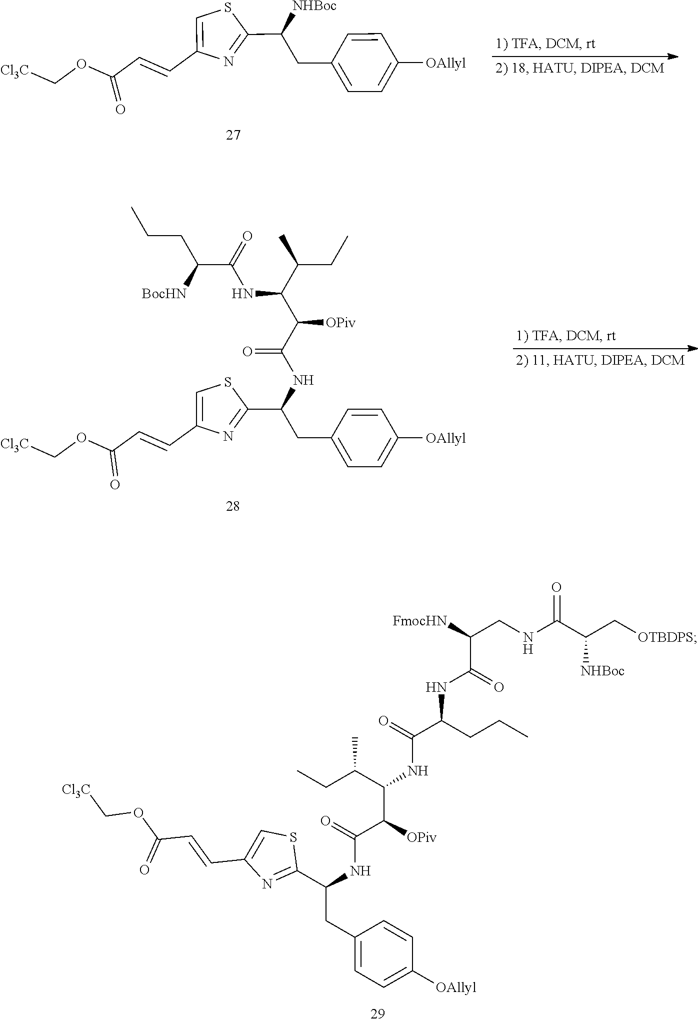

hydrolyzing a trichloroethyl ester group of the compound 29 in an acetic acid solution with zinc (Zn) dust to obtain a second hydrolyzed compound, removing a Boc protection group from the second hydrolyzed compound with the trifluoroacetic acid to obtain a sixth group-removed compound, and performing an intra-molecular macrocyclic-closing reaction on the sixth group-removed compound in a dichloromethane (DCM) solution with 2-(7-azabenzotriazol-1-yl)-N,N,N′,N′-tetramethyluronium hexafluorophosphate (HATU) to obtain the compound 2;
where a specific synthesis process of the compound 2 is as follows:

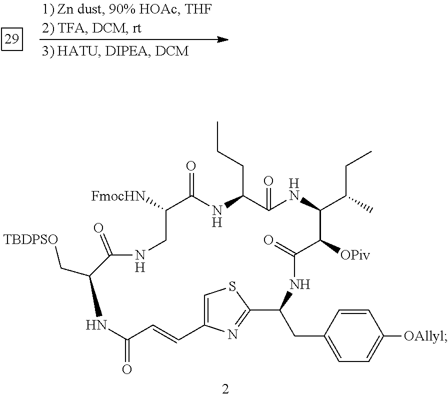

where structures of the compound 11 and the compound 18 are as follows:

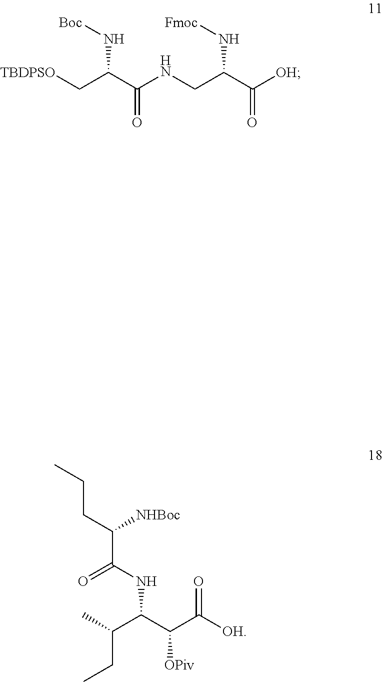

In an embodiment, a synthetic method of the compound 18 includes:
taking a compound 12 as a starting material, performing a methyl esterification on the compound 12 to obtain a methyl esterified compound, protecting an amino group of the methyl esterified compound with di-tert-butyl dicarbonate to obtain a first intermediate compound, and reducing a methyl ester group of the first intermediate compound with the NaBH₄ to obtain a compound 13; oxidizing a primary alcohol group of the compound 13 with the IBX to obtain an aldehyde, performing a reaction between the aldehyde and acetone cyanohydrin to obtain a second intermediate compound, refluxing and hydrolyzing, by using a MeOH solution of hydrogen chloride, the second intermediate compound without purifying to obtain a third hydrolyzed compound, and protecting an amino group of the third hydrolyzed compound with the di-tert-butyl dicarbonate to obtain two diastereoisomers, a compound 14 and a compound 14′;
where a specific synthesis process of the compound 14 and the compound 14′ is as follows:

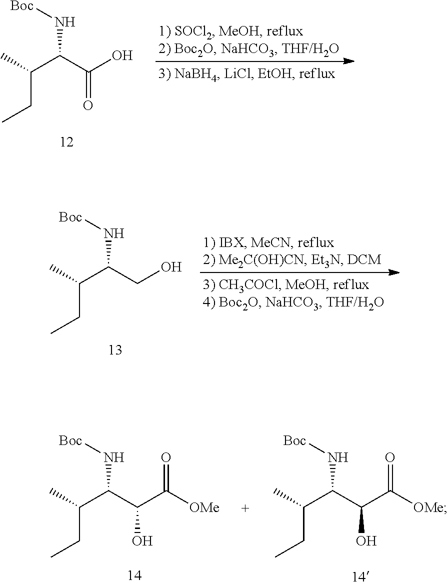 [SMILES: CC[C@H](C)[C@H](NC(=O)OC(C)(C)C)C(=O)O ; CC[C@H](C)[C@@H](CO)NC(=O)OC(C)(C)C ; CC[C@H](C)[C@H](NC(=O)OC(C)(C)C)[C@@H](O)C(=O)OC ; CC[C@H](C)[C@H](NC(=O)OC(C)(C)C)[C@H](O)C(=O)OC]

performing a reaction on the compound 14 and pivaloyl chloride (CsH₉ClO) to obtain a compound 15, removing a Boc protection group of the compound 15 with the trifluoroacetic acid to obtain a seventh group-removed compound, and preforming a peptide grafting reaction between the seventh group-removed compound and a compound 16 to obtain a compound 17; and hydrolyzing a methyl ester group of the compound 17 in a reflux condition of pyridine with lithium iodide to obtain the compound 18;
where a specific synthesis process of the compound 18 is as follows:

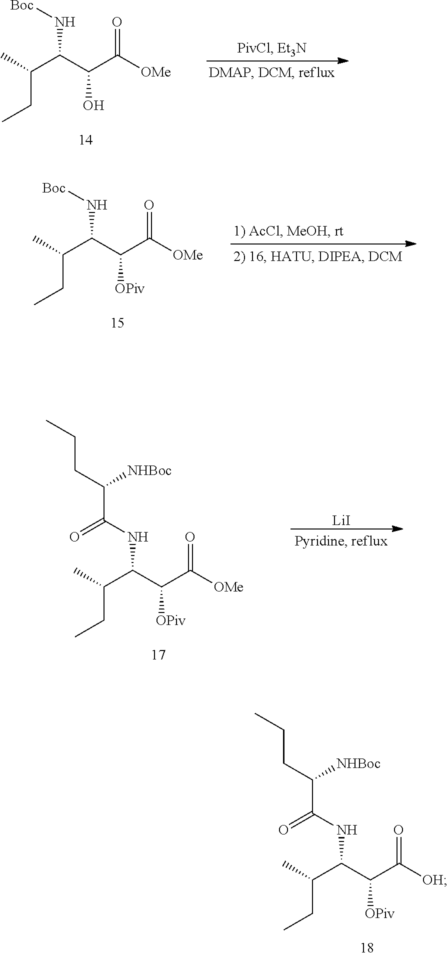

and
where a structure of the compound 16 is as follows:

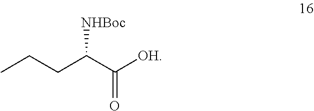

In an embodiment, a synthetic method of the compound 11 includes:
removing a Boc protection group of the compound 8 with the trifluoroacetic acid to obtain an eighth group-removed compound, and preforming a peptide grafting reaction on the eighth group-removed compound and a compound 9 to obtain a compound 10; and removing a phenoxyacetyl (Pac) protection group from the compound 10 in the acetic acid solution with the zinc dust to obtain the compound 11;
where a specific synthesis process of the compound 11 is as follows:

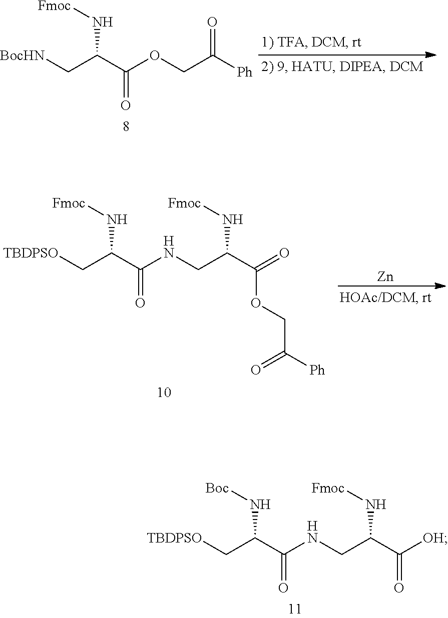

where a structure of the compound 9 is as follows:

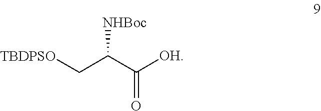

In an embodiment, a synthetic method of the compound 8 includes:
taking a compound 6 as a starting material, protecting an amino group of the compound 6 with N-(9-fluorenylmethoxycarbonyloxy)succinimide (Fmoc-Osu) to obtain a compound 7, and performing a Hofmann rearrangement reaction on the compound 7 to obtain a compound 3; and protecting an amino group of the compound 3 with the di-tert-butyl dicarbonate (BOC₂O), and protecting a carboxyl group of the compound 3 with phenacyl bromide (PacBr), to obtain the compound 8;
where a specific synthesis process of the compound 8 is as follows:

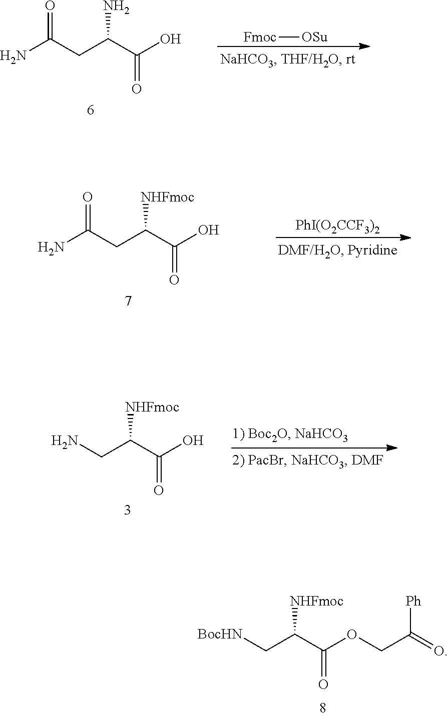

According to a third aspect, an intermediate compound is provided, and a specific structure of the intermediate compound is as follows:

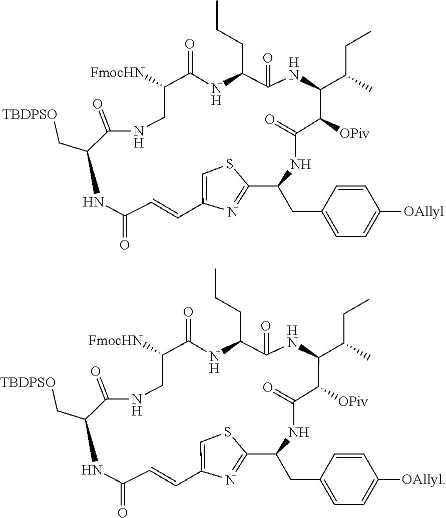

According to a fourth aspect, a pharmaceutical composition is provided, and the pharmaceutical composition includes anyone of the compounds or the pharmaceutically acceptable salts thereof as described in the first aspect.

According to a fifth aspect, an application method of anyone of the compounds or the pharmaceutically acceptable salts thereof as described in the first aspect, and the application method includes:
applying the compound or the pharmaceutically acceptable salt thereof to prepare a protease inhibitor.

Beneficial effects of the technical solutions provided by the embodiments of the disclosure are as follows: the CyclotheonellazoleA natural product is successfully synthesized in the disclosure by using a classical retrosynthesis analysis, a stereoscopic structure of the CyclotheonellazoleA natural product is confirmed, extremely excellent protease inhibitory activity of the CyclotheonellazoleA natural product is confirmed by combining a protease inhibition activity experiment, and the CyclotheonellazoleA has a strong application prospect in a pharmaceutical industry.

### BRIEF DESCRIPTION OF DRAWINGS

In order to provide a clearer explanation of technical solutions in embodiments of the disclosure, drawings required in descriptions of the embodiments will be simply introduced below. Apparently, the drawings described below are merely some of the embodiments of the disclosure, for those skilled in the art, other drawings can be obtained according to the drawings without creative work.

FIG. 1 illustrates a schematic diagram of an enzyme activity of transmembrane serine protease 2 (TMPRSS2) under an inhibiting of CyclotheonellazoleA (CA) according to an embodiment of the disclosure.

FIG. 2 illustrates a schematic diagram of an enzyme activity of mainprotease (Mpro) of SARS-COV-2 virus under the inhibiting of the CA according to an embodiment of the disclosure.

FIG. 3 illustrates a schematic diagram of an enzyme activity of neutrophil elastase (NE) under the inhibiting of the CA according to an embodiment of the disclosure.

### DETAILED DESCRIPTION OF EMBODIMENTS

In order to make purposes, technical solutions and advantages of the disclosure more clearly, embodiments of the disclosure will be further described in conjunction with drawings.

### Embodiment 1 a Synthesis of a CyclotheonellazoleA (i.e., a Compound 1, CA)

A retrosynthesis analysis of the compound CA is as follows:

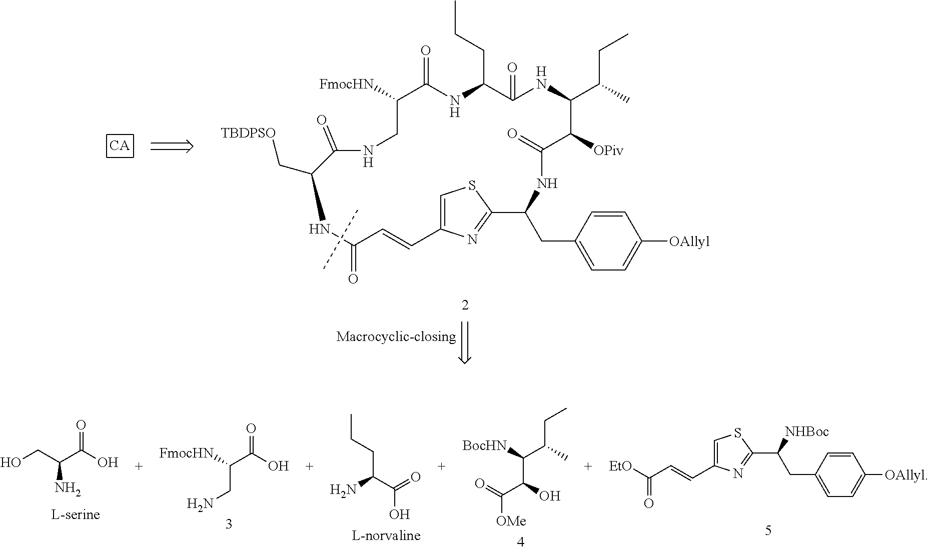

(1) A synthesis process of a compound 8 is as follows:

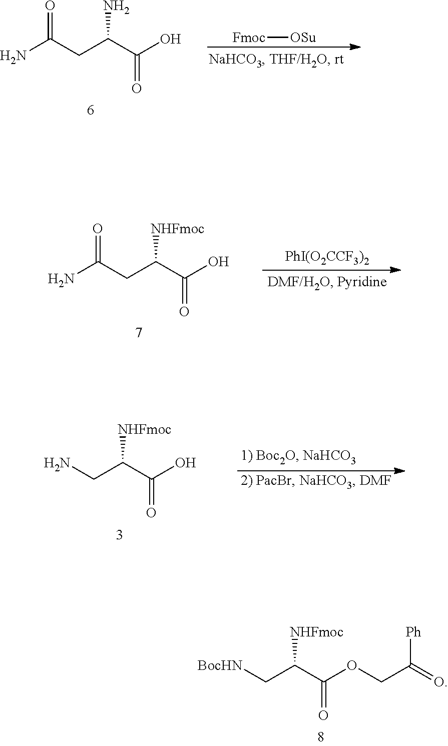

L-asparagine (i.e., a compound 6) (20 grams abbreviated as g, 151.4 micromolar abbreviated as mmol) is dissolved in a mixed solvent of tetrahydrofuran (THF)/water (1:1, 1000 millimeters abbreviated as mL), sodium bicarbonate (NaHCO₃) solid (33.6 g, 400 mmol) is added, after stirring evenly, N-(9-fluorenylmethoxycarbonyloxy)succinimide (Fmoc-OSu) (51.0 g, 151.4 mmol) is added to obtain a mixture. After stirring of the mixture for reacting at a room temperature for 15 hours (h), the mixture is diluted by adding water (1000 mL) to obtain a diluted solution. Hydrochloric acid is added to acidify the diluted solution to make a potential of hydrogen (pH) thereof be equal to 2, and a large amount of white solids are precipitated. The white solids are filtered to obtain a filter residue, the filter residue is washed by a large amount of water (2000 mL) to obtain solids, and the solids are dried in an oven to obtain a compound 7.

The compound 7 is dissolved in a mixed solvent of N,N-dimethylformamide (DMF)/water (2:1, 600 mL), after cooling to a temperature of 0 Celsius degree (° C.), [bis(trifluoroacetoxy)iodo]benzene (PhI(O₂CCF₃)₂) (72.0 g, 166.5 mmol) is added, after stirring for 15 minutes (min), pyridine (24.5 mL, 303 mmol) is added to obtain a mixture. After 30 min, the mixture is stirred for reacting at the room temperature for 15 h to obtain a reaction solution. The reaction solution is concentrated under reduced pressure to remove all solvents therein, diluted by adding water (500 mL), and acidified to make pH=3 by adding hydrochloric acid to thereby obtain an aqueous phase. The aqueous phase is washed for twice with ethyl acetate (EA) (200 mL) to obtain washed aqueous phase (i.e., a compound 3).

NaHCO₃ solid is slowly added into the washed aqueous phase (i.e., the compound 3) to neutralize the washed aqueous phase to make pH=8, and THF (500 mL) and di-tert-butyl dicarbonate ((Boc)₂O) (34.5 mL, 151.0 mmol) are added to obtain a mixture. After stirring of the mixture for reacting at the room temperature for 10 h, the mixture is concentrated under reduced pressure to remove THF therein, acidified to make pH=4 by adding hydrochloric acid, and extracted for three times with EA (500 mL) to obtain organic phases. The organic phases are merged, dried with anhydrous sodium sulfate (Na₂SO₄), and concentrated under reduced pressure to obtain an intermediate.

The intermediate is dissolved in DMF (150 mL), NaHCO₃ solid (33.6 g, 400 mmol) and 2-bromoacetophenone (PacBr) (30.0 g, 150.0 mmol) are added to obtain a mixture. After stirring of the mixture for reacting at the room temperature for 8 h, the mixture is concentrated under reduced pressure to remove solvents therein, diluted by adding water (500 mL) and extracted for twice with EA (500 mL) to obtain organic phases. The organic phases are merged, dried with anhydrous Na₂SO₄, and concentrated under reduced pressure to obtain a crude product. Petroleum ether (PE) and EA with a ratio of 2:1 are used as an eluent to perform a flash column chromatography on the crude product to obtain the compound 8 (a white solid, 66.0 g), a total yield is 80% through the above four steps.

The compound 8 is detected through hydrogen-1 nuclear magnetic resonance (¹HNMR), carbon-13 nuclear magnetic resonance (¹³CNMR) and high-resolution mass spectrometry (HRMS), the compound 8 is a pure compound, and performance indicators or characterization data of the compound 8 are as follows: [α]²_{D}⁵-70.2(c 0.82, CHCl₃); ¹H NMR(400 MHZ, CDCl₃) δ 7.90(d, J=7.7 Hz, 2H), 7.74(d, J=7.5 Hz, 2H), 7.61(d, J=6.0 Hz, 3H), 7.49(t, J=7.6 Hz, 2H), 7.38(t, J=7.4 Hz, 2H), 7.28(dd, J=12.9, 5.8 Hz, 2H), 6.36(s, 1H), 5.82(s, 1H), 5.67(d, J=16.5 Hz, 1H), 5.24(d, J=16.5 Hz, 1H), 4.64-4.49(m, 1H), 4.34(dd, J=15.1, 8.6 Hz, 2H), 4.24(t, J=7.1 Hz, 1H), 3.89(s, 1H), 3.68(d, J=14.1 Hz, 1H), 1.48(s, 9H); ¹³C NMR (100 MHz, CDCl₃) δ 192.17, 170.01, 157.23, 156.06, 143.79, 141.12, 134.20, 133.57, 128.87, 127.72, 127.55, 126.96, 125.16, 119.80, 79.89, 67.13, 66.52, 55.49, 46.97, 42.17, 28.21; HR-ESIMS m/z: calculated for C₃₁H₃₂N₂O₇Na⁺[M+Na]⁺: 567.2210, found 567.2214.

(2) A synthesis process of a compound 11 is as follows:

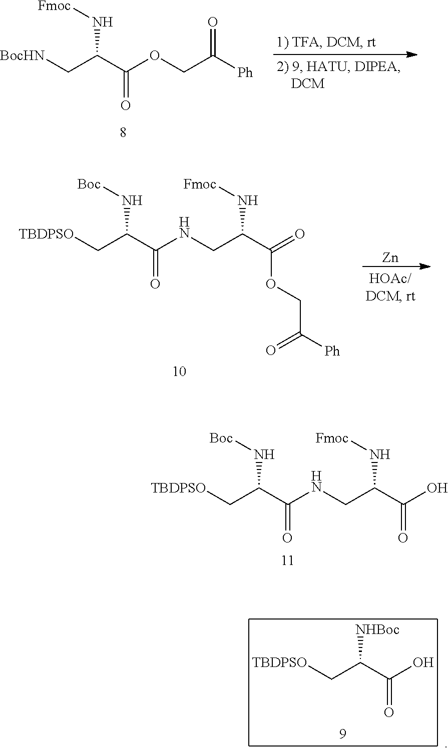

The compound 8 (23.4 g, 42.9 mmol) is dissolved in dichloromethane (DCM) solution (200 mL), trifluoroacetic acid (TFA) (40 mL) is added to obtain a mixture. After stirring of the mixture for reacting at the room temperature for 2 h, the mixture is concentrated under reduced pressure and dried fully to obtain an intermediate amine (i.e., an eighth group-removed compound).

The intermediate amine is dissolved in anhydrous DCM (500 mL), 2-(7-azabenzotriazol-1-yl)-N,N,N′,N′-tetramethyluronium hexafluorophosphate (HATU) (20.0 g, 52.0 mmol) and a compound 9 (18.6 g, 42 mmol) are sequentially added. After cooling to the temperature of 0° C., N,N-diisopropylethylamine (DIPEA) (21.5 mL, 128.7 mmol) is slowly added dropwise to react for 30 min to obtain a mixture. After heating of the mixture to the room temperature for reacting for 12 h, an organic phase of the mixture is sequentially washed by diluted hydrochloric acid (300 mL) with a concentration of 2 molar per liter (M), and washed by saturated NaHCO₃ solution (300 mL) to thereby obtain an organic phase. The organic phase is dried with anhydrous Na₂SO₄, and concentrated under reduced pressure to obtain a crude product, PE and EA with the ratio of 2:1 are used as an eluent to perform a flash column chromatography on the crude product to obtain a compound 10 (a white solid, 31.0 g), a total yield thereof is 85%.

The compound 10 is detected through ¹HNMR, ¹³CNMR and HRMS, the compound 10 is a pure compound, and performance indicators or characterization data of the compound 10 are as follows: [α]²_{D}⁵-18.2(c1.0, CHCl₃); ¹H NMR(400 MHZ, CDCl₃) δ 7.92(d, J=7.2 Hz, 2H), 7.74(d, J=7.3 Hz, 2H), 7.63(t, J=6.2 Hz, 7H), 7.51(t, J=7.7 Hz, 2H), 7.43-7.30(m, 10H), 6.16(d, J=6.8 Hz, 1H), 5.68(d, J=16.5 Hz, 1H), 5.57(s, 1H), 5.27(d, J=16.4 Hz, 1H), 4.69(s, 1H), 4.31(s, 3H), 4.23(d, J=7.4 Hz, 1H), 4.13-4.10(m, 1H), 3.93-3.83(m, 1H), 3.72(d, J=13.8 Hz, 1H), 1.35(s, 9H), 1.04(s, 9H); ¹³C NMR(100 MHz, CDCl₃) δ 192.29, 171.74, 169.74, 156.14, 155.56, 143.85, 141.16, 135.52, 135.46, 134.78, 134.51, 133.39, 132.96, 132.60, 129.83, 129.42, 128.97, 127.94, 127.76, 127.56, 127.08, 127.04, 125.37, 119.79, 79.99, 67.39, 66.62, 64.13, 60.33, 56.97, 54.59, 47.01, 41.05, 28.19, 26.70, 26.53, 19.23; HR-ESIMS m/z: calculated for C₅₀H₅₅N₃O₉SiNa⁺[M+Na]⁺: 892.3708, found 892.3710.

The compound 10 (31.0 g, 35.6 mmol) is dissolved in DCM solution (500 mL), an acetic acid (HOAc) solution (100 mL) is added, after stirring evenly, zinc (Zn) dust (46.3 g, 712.0 mmol) is added to obtain a mixture. After stirring of the mixture for reacting at the room temperature for 5 h, the mixture is concentrated under reduced pressure to remove DCM therein, and diluted by adding water (800 mL) to obtain a diluted solution, NaHCO₃ solid is slowly added to neutralize the diluted solution to make pH=8 to obtain a neutralized solution. The neutralized solution is extracted with DCM solution (800 mL) and filtered to remove the zinc dust therein to obtain a filtrate. The filtrate is separated into an organic phase and an aqueous phase, the aqueous phase is extracted for twice with DCM solution (500 mL) to obtain organic phases. The organic phases are merged, dried with anhydrous Na₂SO₄, and concentrated under reduced pressure to obtain the compound 11.

(3) A synthesis process of a compound 14 and a compound 14′ is as follows:

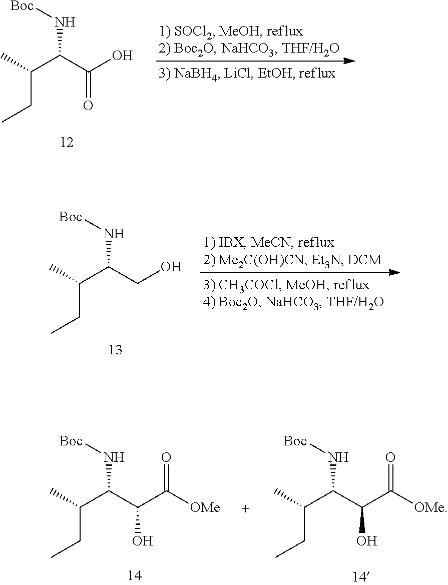 [SMILES: CC[C@H](C)[C@H](NC(=O)OC(C)(C)C)C(=O)O ; CC[C@H](C)[C@@H](CO)NC(=O)OC(C)(C)C ; CC[C@H](C)[C@H](NC(=O)OC(C)(C)C)[C@@H](O)C(=O)OC ; CC[C@H](C)[C@H](NC(=O)OC(C)(C)C)[C@H](O)C(=O)OC]

L-isoleucine (i.e., a compound 12) (20.0 g, 160.0 mmol) is dissolved in methanol solution (MeOH) (200 mL), after cooling to the temperature of 0° C., thionyl chloride (SOCl₂) (23 mL, 320 mmol) is slowly added dropwise to react for 30 min to obtain a mixture. After heating and refluxing of the mixture for reacting for 3 h, the mixture is cooled to the room temperature, and the mixture is concentrated under reduced pressure to obtain a first intermediate (i.e., methyl esterified compound).

The first intermediate obtained in the previous step is dissolved in a mixed solvent of THF/water (1:1, 1000 mL), NaHCO₃ solid (40.3 g, 480 mmol) is added, after stirring evenly, di-tert-butyl decarbonate ((Boc)₂O) (36.8 mL, 160.0 mmol) is added to obtain a mixture. After stirring of the mixture for reacting for 15 h, the mixture is concentrated under reduced pressure to remove THF therein, diluted by adding water (200 mL), and extracted for three times with EA (500 mL) to obtain organic phases. The organic phases are merged, dried with anhydrous Na₂SO₄, and concentrated under reduced pressure to obtain a second intermediate (i.e., a first intermediate compound).

The second intermediate obtained in the previous step is dissolved in ethanol (EtOH) (1000 mL), and a sodium borohydride (NaBH₄) solid (12.2 g, 320 mmol) and anhydrous lithium chloride (LiCI) (13.6 g, 320 mmol) are sequentially added to obtain a mixture. After heating and refluxing of the mixture for reacting for 3 h, the mixture is cooled to the room temperature, and the mixture is concentrated under reduced pressure to remove EtOH therein, to thereby obtain a solid. Water (800 mL) is added to dissolve the solid to obtain a solution. Hydrochloric acid is slowly added dropwise into the solution to make pH=5 to thereby obtain an acidic solution. The acidic solution is extracted for three times by using EA (500 mL) to obtain organic phases. The organic phases are merged, dried with anhydrous Na₂SO₄, and concentrated under reduced pressure to obtain a compound 13 (31.3 g), and a total yield thereof is 90% through the above three steps.

The compound 13 (31.3 g, 144 mmol) is dissolved in acetonitrile (i.e., MeCN) (300 mL), and 2-iodoxybenzoic acid (IBX) (44.8 g, 160 mmol) is added to obtain a mixture. After heating and refluxing of the mixture for reacting for 2 h, the mixture is cooled to the room temperature, and the mixture is filtered to obtain a filter residue and a filtrate. The filter residue is washed for once with acetonitrile (200 mL), and the filtrate is concentrated under reduced pressure to obtain an intermediate aldehyde.

The intermediate aldehyde obtained in the previous step is dissolved in DCM solution (300 mL), and triethylamine (Et₃N) (24.0 mL, 173 mmol) and acetone cyanohydrin (14.6 mL, 160 mmol) are added to obtain a mixture. After stirring of the mixture for reacting at the room temperature for 2 h, the mixture is filtered to obtain a filtrate. The filtrate is concentrated under reduced pressure to obtain an intermediate cyanohydrin (i.e., a second intermediate compound).

The intermediate cyanohydrin obtained in the previous step is dissolved in MeOH solution (300 mL), after cooling to the temperature of 0° C., acetyl chloride (51.0 mL, 720 mmol) is slowly added dropwise to react for 30 min to obtain a mixture. After heating and refluxing of the mixture for reacting for 10 h, the mixture is cooled to the room temperature, and the mixture is concentrated under reduced pressure to obtain a third intermediate (i.e., a third hydrolyzed compound).

The third intermediate obtained in the previous step is dissolved in a mixed solvent of THF/water (1:1, 1000 mL), NaHCO₃ solid (40.3 g, 480 mmol) is added, after stirring evenly, di-tert-butyl decarbonate (33.0 mL, 144.0 mmol) is added to obtain a mixture. After stirring of the mixture for reacting at the room temperature for 10 h, the mixture is concentrated under reduced pressure to remove THE therein, diluted by adding water (200 mL), and extracted for three times with EA (500 mL) to obtain organic phases. The organic phases are merged, dried with anhydrous Na₂SO₄, and concentrated under reduced pressure to obtain a crude product. PE and EA with a ratio of 5:1 are used as an eluent to perform a flash column chromatography on the crude product to obtain the compound 14 (16.8 g) and the compound 14′ (16.8 g), a total yield thereof is 85%.

The compound 14 is detected through ¹HNMR, ¹³CNMR and HRMS, the compound 14 is a pure compound, and performance indicators or characterization data of the compound 14 are as follows: [α]²_{D}⁵-48.6(c1.41, CHCl₃); ¹H NMR(400 MHZ, CDCl₃) δ 4.83(d, J=9.0 Hz, 1H), 4.36(s, 1H), 3.77(s, 3H), 3.32(s, 1H), 1.62(dd, J=15.3, 7.1 Hz, 1H), 1.40(s, 9H), 1.34-1.03(m, 2H), 0.99(d, J=6.5 Hz, 3H), 0.90(t, J=7.4 Hz, 3H); ¹³C NMR(100 MHz, CDCl₃) δ 174.87, 155.46, 79.25, 70.28, 56.96, 52.66, 36.18, 28.19, 25.52, 15.66, 10.92; HR-ESIMS m/z: calculated for C₁₃H₂₅NO₅Na⁺[M+Na]⁺: 298.1733, found 298.1735.

The compound 14′ is detected through ¹HNMR, ¹³CNMR and HRMS, the compound 14′ is a pure compound, and performance indicators or characterization data of the compound 14′ are as follows: [α]²_{D}⁵-8.1(c0.62, CHCl₃); ¹H NMR(400 MHZ, CDCl₃) δ 4.79(d, J=8.7 Hz, 1H), 4.31(m, 1H), 3.79(s, 3H), 3.37(s, 1H), 1.64-1.60(m, 1H), 1.45(d, J=3.9 Hz, 9H), 1.21-1.05(m, 2H), 0.93-0.87(m, 6H); ¹³C NMR(100 MHz, CDCl₃) δ 173.85, 156.18, 79.68, 72.59, 58.01, 52.49, 35.37, 28.30, 28.19, 24.84, 15.94, 11.13; HR-ESIMS m/z: calculated for C₁₃H₂₅NO₅Na⁺[M+Na]⁺: 298.1733, found 298.1735.

(4) A synthesis process of a compound 18 is as follows:

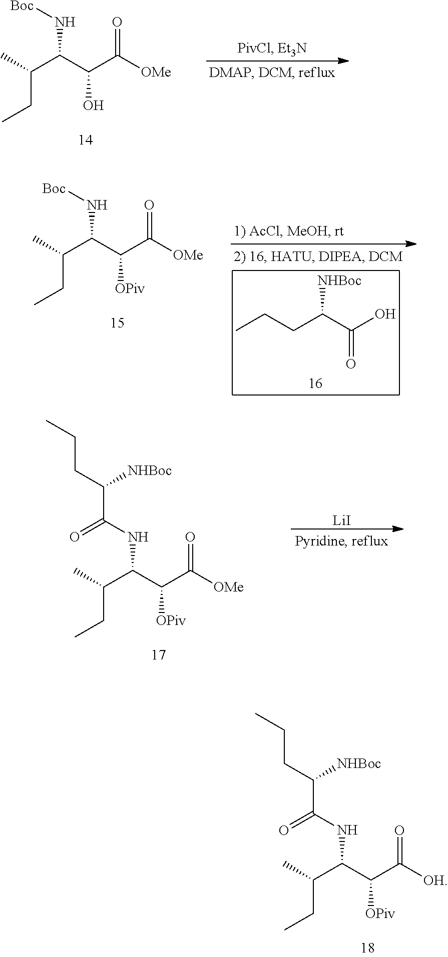

The compound 14 (16.8 g, 61.2 mmol) is dissolved in anhydrous DCM (200 mL), and Et₃N (12.8 mL, 92.0 mmol) and 4-dimethylaminopyridine (DMAP) (0.75 g, 6.1 mmol) are added, after cooling to the temperature of 0° C., pivaloyl chloride (8.3 mL, 67.5 mmol) is slowly added dropwise to react for 30 min to obtain a mixture. After heating and refluxing of the mixture for reacting for 3 h, the mixture is cooled to the room temperature, and the mixture is concentrated under reduced pressure to remove DCM therein to obtain a residue. The residue is dissolved in EA (500 mL) to obtain a residue solution. The residue solution is sequentially washed by diluted hydrochloric acid (150 mL) with a concentration of 1 M, washed by saturated NaHCO₃ solution (200 mL), dried with anhydrous Na₂SO₄, and concentrated under reduced pressure to obtain a compound 15.

The compound 15 obtained in the previous step is dissolved in MeOH solution (150 mL), after cooling to the temperature of 0° C., acetyl chloride (21.8 mL, 306 mmol) is slowly added dropwise to react for 30 min to obtain a mixture. After heating of the mixture to the room temperature for reacting for 2 h, the mixture is concentrated under reduced pressure to obtain an intermediate (i.e., a seventh group-removed compound).

The intermediate obtained in the previous step is dissolved in anhydrous DCM (400 mL), and HATU (30.4 g, 80 mmol) and a compound 16 (13.0 g, 60 mmol) are sequentially added, after cooling to the temperature of 0° C., DIPEA (30.0 mL, 180 mmol) is slowly added dropwise to react for 30 min to obtain a mixture. After heating of the mixture to the room temperature for reacting for 12 h, an organic phase of the mixture is sequentially washed by diluted hydrochloric acid (300 mL) with a concentration of 2 M, washed by using saturated NaHCO₃ solution (300 mL), dried the anhydrous Na₂SO₄, and concentrated under reduced pressure to obtain a crude product. PE and EA with a ratio of 2:1 are used as an eluent to perform a flash column chromatography on the crude product to obtain a compound 17 (23.4 g), and a total yield thereof is 85% through the above three steps.

The compound 17 is detected through ¹HNMR, ¹³CNMR and HRMS, the compound 17 is a pure compound, and performance indicators or characterization data of the compound 17 are as follows: [α]²_{D}⁵-50.7(c0.67, CHCl₃); ¹H NMR(400 MHZ, CDCl₃) δ 6.31(d, J=9.7 Hz, 1H), 5.15(d, J=2.0 Hz, 1H), 5.07(dd, J=15.5, 6.3 Hz, 1H), 4.35-4.29(m, 1H), 4.02-3.96(m, 1H), 3.67(s, 3H), 1.74(d, J=7.1 Hz, 1H), 1.52-1.46(m, 4H), 1.41(s, 9H), 1.32(d, J=7.4 Hz, 4H), 1.26(s, 9H), 1.16(s, 2H), 0.92-0.87(m, 9H); ¹³C NMR(100 MHz, CDCl₃) δ 177.27, 171.94, 168.80, 155.86, 79.98, 71.37, 54.27, 53.48, 52.30, 38.83, 36.53, 28.21, 27.09, 26.96, 25.35, 18.71, 15.30, 13.63, 10.80; HR-ESIMS m/z: calculated for C₂₃H₄₂N₂O₇Na⁺[M+Na]⁺: 480.2992, found 480.2994.

The compound 17 (23.4 g, 51.0 mmol) is dissolved in anhydrous pyridine (200 mL), and anhydrous lithium iodide (13.7 g, 102 mmol) is added to obtain a mixture. After heating and refluxing of the mixture for reacting for 8 h, the mixture is concentrated under reduced pressure to remove pyridine therein, diluted by adding water (500 mL), acidified by adding concentration hydrochloric acid to make pH=4, and extracted for three times with EA (400 mL) to obtain organic phases. The organic phases are merged, dried with anhydrous Na₂SO₄, and concentrated under reduced pressure to obtain a compound 18, and a crude yield thereof is 100%.

(5) A synthesis process of a compound 21 is as follows:

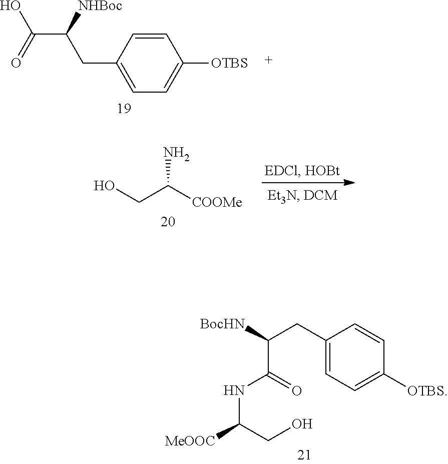

A compound 19 (23.3 g, 82.8 mmol) and a compound 20 (15.5 g, 99.4 mmol, 1.2 equivalent abbreviated as eq) are mixed to obtain a first mixture, and the first mixture is dissolved in DCM solution (200 mL), and Et₃N (45.8 mL, 331.2 mmol, 4.0 eq), 1-ethyl-3(3-dimethylpropylamine) carbodiimide (EDCI) (20.6 g, 107.6 mmol, 1.3 eq), and 1-hydroxybenzotriazole (HOBT) (5.6 g, 41.4 mmol, 0.5 eq) are slowly added to obtain a second mixture. After reacting of the second mixture at the room temperature for 24 h, the second mixture is washed by adding water (200 mL), and separated to obtain an organic phase. The organic phase is concentrated to obtain a crude product. The crude product is dissolved in EA (200 mL) to obtain a crude product solution. The crude product solution is washed by hydrochloric acid with a concentration of 2 normality (N) to obtain a washed crude product solution. An organic phase of the washed crude product solution is washed by saturated salt water, dried with anhydrous Na₂SO₄, filtered, and concentrated to obtain the compound 21 (27.2 g, a total yield of 86%).

The compound 21 is detected through ¹HNMR, ¹³CNMR and HRMS, the compound 21 is a pure compound, and performance indicators or characterization data of the compound 21 are as follows: ¹H NMR(400 MHZ, CDCl₃) δ 7.50(s, 1H), 6.98(dt, J=7.6, 1.1 Hz, 2H), 6.80(t, J=5.5 Hz, 1H), 6.77-6.72(m, 2H), 6.24(s, 1H), 4.95(t, J=7.1 Hz, 1H), 4.64-4.56(m, 2H), 4.30(t, J=6.9 Hz, 1H), 4.16(ddd, J=12.4, 7.0, 5.4 Hz, 1H), 3.63(s, 3H), 3.32(ddt, J=12.5, 7.2, 1.1 Hz, 1H), 3.01(ddt, J=12.5, 7.2, 1.1 Hz, 1H), 1.44(s, 9H); ¹³C NMR(101 MHz, CDCl₃) δ 172.52, 171.89, 156.33, 155.69, 130.58, 128.85, 115.82, 79.69, 62.47, 56.85, 55.27, 53.19, 37.95, 28.34; HRMS(m/z): calculated for C₁₈H₂₆N₂NaO₇⁺([M+Na]⁺): 405.1632, found 405.1622.

(6) A synthesis process of a compound 22 is as follows:

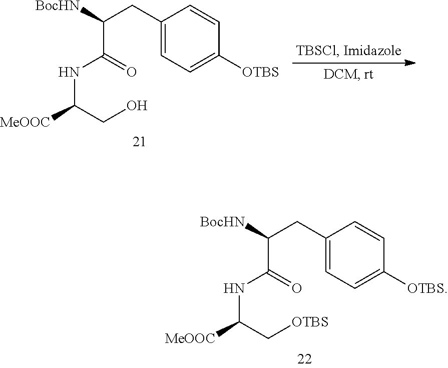

The compound 21 (27.2 g, 71.1 mmol) is dissolved in DCM solution (200 mL), and tert-butyldimethylsilyl chloride (TBSCL) (23.6 g, 156.4 mmol, 2.2 eq) and imidazole (12.1 g, 177.8 mmol, 2.5 eq) are added to obtain a mixture. After reacting of the mixture at the room temperature for 4 h, the mixture is quenched by adding water (200 mL), washed, separated, and concentrated to obtain a crude product. A flash column chromatography with a silica gel pad is performed on the crude product to obtain the compound 22 (43.4 g, a total yield of 100%).

The compound 22 is detected through ¹HNMR, ¹³CNMR and HRMS, the compound 22 is a pure compound, and performance indicators or characterization data of the compound 22 are as follows: ¹H NMR(400 MHZ, CDCl₃) δ 7.50(s, 1H), 7.13(dt, J=7.5, 1.1 Hz, 2H), 7.00-6.94(m, 2H), 6.24(s, 1H), 4.98(dt, J=19.8, 7.0 Hz, 2H), 4.53(dd, J=12.4, 7.1 Hz, 1H), 4.28(dd, J=12.4, 7.1 Hz, 1H), 3.63(s, 3H), 3.49(ddt, J=12.5, 7.0, 1.1 Hz, 1H), 3.23(ddt, J=12.4, 7.2, 1.1 Hz, 1H), 1.44(s, 9H), 1.03(s, 9H), 0.98(s, 9H), 0.21(s, 6H), 0.08(s, 6H); ¹³C NMR (101 MHz, CDCl₃) δ 171.89, 170.44, 155.69, 154.23, 130.44, 129.76, 119.74, 79.69, 65.53, 56.85, 54.42, 53.19, 37.95, 28.34, 25.87, 25.43, 18.27, 18.22, −4.39, −5.44; HRMS(m/z): calculated for C₃₀H₅₄N₂NaO₇Si₂⁺([M+Na]⁺): 633.3362, found 633.3351.

(7) A synthesis process of a compound 24 is as follows:

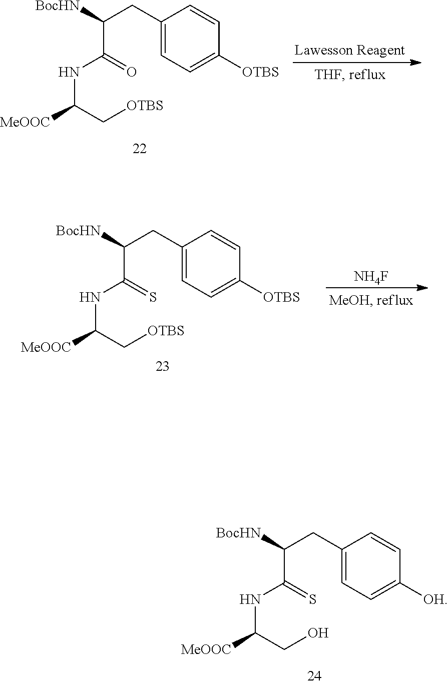

The compound 22 (43.4 g, 71.1 mmol) is dissolved in an anhydrous THF (200 mL) solution, and a Lawesson's reagent (34.5 g, 85.3 mmol, 1.2 eq) is added to obtain a mixture. After reacting of the mixture at a temperature of 70° C. for 24 h, and restoring to the room temperature, the mixture is quenched by adding water (150 mL), concentrated to remove THF therein, and extracted and concentrated with EA to obtain a crude product compound 23.

A MeOH solution (100 mL) of the crude product compound 23 is prepared, NH₄F solid (13.2 g, 355.5 mmol, 5.0 eq) is added to obtain a mixture. After reacting of the mixture at a temperature of 80° C. for 2 h, stop heating and restoring to the room temperature, the mixture is quenched by adding water (100 mL), concentrated to remove MeOH therein, extracted with EA, and concentrated to obtain a crude product. The crude product is purified through a column chromatography (PE/EA=2:1 to 1:1, volume/volume abbreviated as V/V) to obtain the compound 24 (19.8 g, a total yield of 70%).

The compound 24 is detected through ¹HNMR, ¹³CNMR and HRMS, the compound 24 is a pure compound, and performance indicators or characterization data of the compound 24 are as follows: ¹H NMR(400 MHZ, CDCl₃) δ 7.34(s, 1H), 7.13(dt, J=7.4, 1.1 Hz, 2H), 6.81-6.70(m, 2H), 5.83(t, J=7.0 Hz, 1H), 5.74(s, 1H), 5.18(t, J=7.1 Hz, 1H), 4.64(s, 1H), 4.40(ddd, J=12.6, 7.1, 5.6 Hz, 1H), 4.20(ddd, J=12.5, 7.1, 5.5 Hz, 1H), 3.63(s, 3H), 3.06(ddt, J=12.5, 7.0, 1.0 Hz, 1H), 2.72(ddt, J=12.5, 7.2, 1.1 Hz, 1H), 1.52(t, J=5.5 Hz, 1H), 1.44(s, 9H); ¹³C NMR(101 MHz, CDCl₃) δ 200.49, 170.97, 156.87, 156.33, 130.63, 130.02, 115.82, 79.69, 63.08, 62.84, 55.30, 53.19, 37.71, 28.34; HRMS(m/z): calculated for C₁₈H₂₆N₂NaO₆S⁺([M+Na]⁺): 421.1404, found 421.1386.

(8) A synthesis process of a compound 25 is as follows:

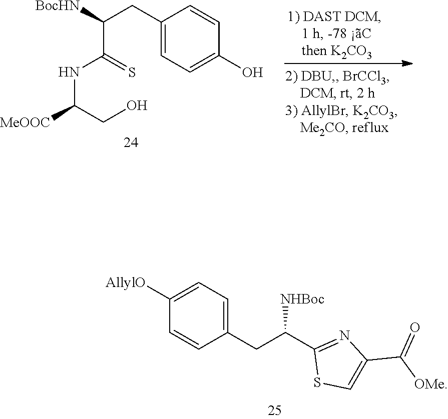

Under a nitrogen protection, the compound 24 (19.8 g, 49.7 mmol) is dissolved in anhydrous THF (80 mL), after cooling to a temperature of −50° C., diethylaminosulfur trifluoride (DAST) (16.5 mL, 125 mmol, 2.5 eq) is slowly added dropwise, and then stirring is performed for 2 h to obtain a system. NaHCO₃ solid (12.5 g, 149.1 mmol, 3.0 eq) is added into the system, after restoring naturally to the temperature and stirring for 1 h, the system is quenched by adding saturated NaHCO₃ solution, concentrated to remove THF therein, washed and extracted by adding DCM solution, dried with anhydrous Na₂SO₄ solid, filtered, concentrated under reduce pressure to obtain a first intermediate (i.e., a ring-closes compound).

The first intermediate obtained in the previous step is dissolved in anhydrous DCM solution (200 mL), and 1,8-diazabicyclo[5.4.0]undec-7-ene (DBU) (14.8 mL, 99.4 mmol, 2.0 eq) and bromotrichloro methane (CBrCl3) (9.9 mL, 99.4 mmol, 2.0 eq) are added to obtain a mixture. After reacting of the mixture at the room temperature for 2 h, the mixture is concentrated to obtain a crude product. The crude product is dissolved in EA solution (100 mL) to obtain a crude product solution. The crude product solution is washed for three times by hydrochloric acid with a concentration of 2 N to obtain a washed crude product solution. An organic phase of the washed crude product solution is dried with anhydrous Na₂SO₄, filtered, and concentrated to obtain a second intermediate (16.2 g) (i.e., a first oxidized compound).

The second intermediate (16.2 g, 42.8 mmol) obtained in the previous step is dissolved in acetone solution (100 mL), and potassium carbonate (K₂CO₃) (17.7 g, 128.4 mmol, 3.0 eq) and allyl bromide (9.4 mL, 85.6 mmol, 2.0 eq) are added to obtain a mixture. After reacting of the mixture at a temperature of 60° C. for 12 h, and restoring to the room temperature, the mixture is directly filtered and concentrated to obtain the compound 25 (15.8 g, a total yield of 88%).

The compound 25 is detected through ¹HNMR, ¹³CNMR and HRMS, the compound 25 is a pure compound, and performance indicators or characterization data of the compound 25 are as follows: ¹H NMR(400 MHZ, CDCl₃) δ 8.10(s, 1H), 7.21(dt, J=7.6, 1.1 Hz, 2H), 6.93-6.80(m, 2H), 6.06(dtd, J=16.8, 9.7, 2.7 Hz, 1H), 5.88(t, J=7.0 Hz, 1H), 5.59(ddd, J=13.8, 10.0, 1.8 Hz, 1H), 5.34(ddd, J=16.7, 13.9, 1.8 Hz, 1H), 5.19(s, 1H), 4.90-4.75(m, 2H), 3.96(s, 3H), 3.41(ddt, J=12.6, 7.0, 1.1 Hz, 1H), 3.18(ddt, J=12.5, 7.0, 1.0 Hz, 1H), 1.44(s, 9H); ¹³C NMR (101 MHz, CDCl₃) δ 169.83, 162.00, 157.94, 156.87, 146.40, 132.73, 131.05, 130.57, 127.12, 117.83, 115.06, 79.69, 67.72, 53.94, 52.30, 40.65, 28.34; HRMS(m/z): calculated for C₂₁H₂₆N₂NaO₅S⁺([M+Na]⁺): 441.1455, found 441.1445.

(9) A synthesis process of a compound 26 is as follows:

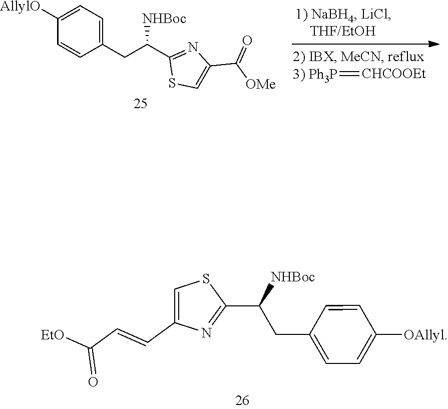

The compound 25 (15.8 g, 37.8 mmol) is dissolved in anhydrous THE solution (80 mL), and LiCl (6.4 g, 151.2 mmol, 4.0 eq) and NaBH₄ solid (5.7 g, 151.2 mmol, 4.0 eq) are added to obtain a system. EtOH solution (90 mL) is added into the system to obtain a mixture. After reacting of the mixture at a temperature of 60° C. for 18 h, and restoring to the room temperature, the mixture is quenched by adding water (90 mL), stirred until the mixture clarifies, concentrated to remove THF and EtOH therein, extracted by adding EA, dried with anhydrous Na₂SO₄ solid, filtered, concentrated to obtain a first intermediate (i.e., a reduced compound).

The first intermediate obtained in the previous step is dissolved in methyl sulfoxide (DMSO) (40 mL), and IBX (10.2 g, 36.6 mmol, 1.2 eq) is added to obtain a mixture. After reacting of the mixture at the room temperature for 1 h, the mixture is washed by adding water (100 mL), extracted by adding EA (80 mL) and filtered to obtain a filtrate. The filtrate is stratified by standing to obtain an aqueous phase and an organic phase. The aqueous phase is extracted for three times with EA to obtain organic phases. The organic phases are merged, washed by saturated salt water, dried with anhydrous Na₂SO₄, filtered, and concentrated under reduced pressure to obtain a second intermediate (i.e., a second oxidized compound).

The second intermediate obtained in the previous step is dissolved in anhydrous DCM (150 mL), and a Wittig reagent (9.2 g, 27.7 mmol, 1.1 eq) is added to obtain a mixture. After reacting of the mixture at the room temperature for 1 h, silica gel is added into the mixture to perform a column chromatography with a dry sampling method (PE/EA=4:1, V/V) on the mixture to obtain the compound 26 (10.7 g, a total yield of 93%).

The compound 26 is detected through ¹HNMR, ¹³CNMR and HRMS, the compound 26 is a pure compound, and performance indicators or characterization data of the compound 26 are as follows: ¹H NMR(400 MHZ, CDCl₃) δ 7.93(s, 1H), 7.71(d, J=15.0 Hz, 1H), 7.28(dt, J=7.6, 1.1 Hz, 2H), 6.90-6.82(m, 2H), 6.28(d, J=15.2 Hz, 1H), 6.06(dtd, J=16.8, 9.9, 2.3 Hz, 1H), 5.66(t, J=7.0 Hz, 1H), 5.59(ddd, J=13.9, 10.1, 1.9 Hz, 1H), 5.41(s, 1H), 5.35(dd, J=16.8, 1.9 Hz, 1H), 4.89-4.82(m, 1H), 4.77(dd, J=12.3, 9.7 Hz, 1H), 4.33(dq, J=11.9, 6.0 Hz, 1H), 4.24(dq, J=12.2, 6.0 Hz, 1H), 3.28(ddt, J=12.3, 7.0, 1.0 Hz, 1H), 3.00(ddt, J=12.3, 7.0, 1.0 Hz, 1H), 1.44(s, 9H), 1.31(t, J=6.0 Hz, 3H); ¹³C NMR(101 MHz, CDCl₃) δ 170.13, 166.95, 157.94, 156.87, 150.72, 133.80, 132.73, 131.05, 130.57, 123.07, 117.83, 117.27, 115.06, 79.69, 67.72, 60.58, 53.94, 40.65, 28.34, 14.31; HRMS(m/z): calculated for C₂₄H₃₀N₂NaO₅S⁺([M+Na]⁺): 481.1768, found 481.1748.

(10) A synthesis process of a compound 27 is as follows:

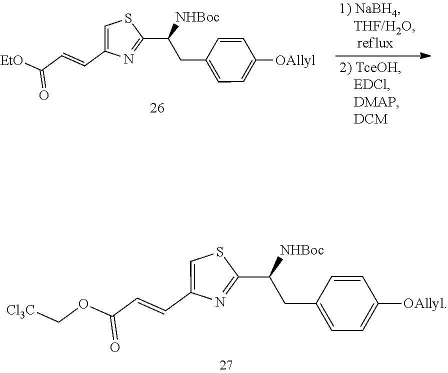

The compound 26 (10.7 g, 23.3 mmol) is dissolved in THF (50 mL), and a sodium hydroxide (NaOH) (1.9 g, 46.6 mmol, 2.0 eq) aqueous solution (50 mL) and EtOH (50 mL) are added to obtain a mixture. After reacting of the mixture at the room temperature for 4 h, the mixture is concentrated to remove the solvent therein, and acidified by hydrochloric acid with a concentration of 2 N to adjust pH to 4, to thereby precipitate a solid. The solid is filtered, washed by distilled water, and dried in an oven at a temperature of 80° C. for 12 h to obtain a white solid (i.e., a first hydrolyzed compound).

The white solid is dissolved in anhydrous DCM (100 mL), and trichloroethanol (3.3 mL, 35 mmol, 1.5 eq), EDCI (8.9 g, 46.6 mmol, 2.0 eq) and DMAP (2.8 g, 23.3 mmol, 1.0 eq) are added to obtain a mixture. After reacting of the mixture at the room temperature for 12 h, the mixture is concentrated to remove DCM therein to obtain an intermediate. The intermediate is dissolved in EA to obtain an intermediate solution. The intermediate solution is washed by hydrochloric acid with a concentration of 2 N to thereby obtain an organic phase. The organic phase is washed by saturated salt water, dried with anhydrous Na₂SO₄, filtered, and precipitated to obtain the compound 27 (12.6 g, a total yield of 96%).

The compound 27 is detected through ¹HNMR, ¹³CNMR and HRMS, the compound 27 is a pure compound, and performance indicators or characterization data of the compound 27 are as follows: ¹H NMR(400 MHZ, CDCl₃) δ 7.93(s, 1H), 7.81(d, J=15.0 Hz, 1H), 7.09(dt, J=7.6, 1.1 Hz, 2H), 6.82-6.74(m, 2H), 6.42(d, J=15.0 Hz, 1H), 6.06(dtd, J=16.8, 9.9, 2.1 Hz, 1H), 5.87(t, J=7.1 Hz, 1H), 5.59(ddd, J=13.7, 10.1, 1.9 Hz, 1H), 5.38-5.27(m, 3H), 5.19(s, 1H), 4.91(dq, J=12.5, 2.0 Hz, 1H), 4.73(dd, J=12.4, 10.0 Hz, 1H), 3.31-3.21(m, 1H), 3.08(ddt, J=12.5, 7.0, 1.1 Hz, 1H), 1.44(s, 9H); ¹³C NMR(101 MHz, CDCl₃) δ 170.13, 168.82, 157.94, 156.87, 150.72, 133.80, 132.73, 131.05, 130.57, 123.07, 117.83, 117.27, 115.06, 95.14, 79.69, 73.88, 67.72, 53.94, 40.65, 28.34; HRMS(m/z): calculated for C₂₄H₂₇C₁₃N₂NaO₅S⁺([M+Na]⁺): 583.0598, found 583.0588.

(11) A synthesis process of a compound 28 is as follows:

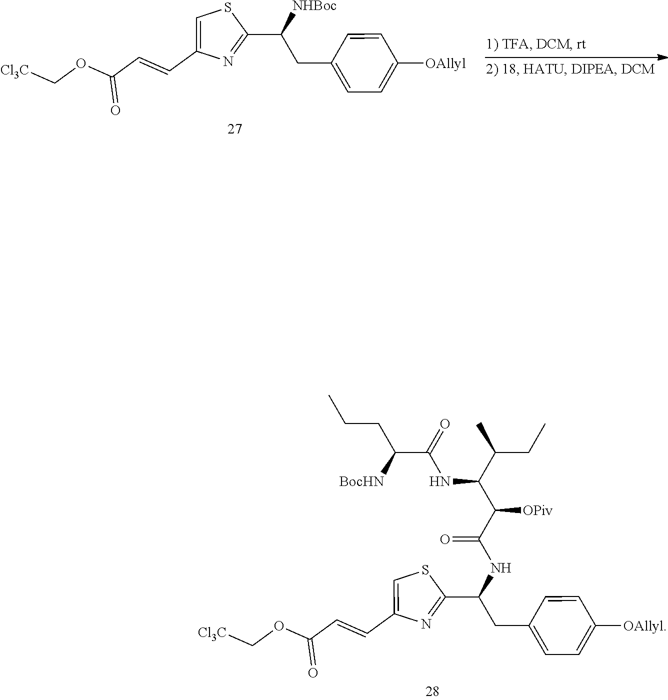

The compound 27 (15.0 g, 26.7 mmol) is dissolved in DCM solution (200 mL), and trifluoroacetic acid (30 mL) is added to obtain a mixture. After stirring of the mixture for reacting at the room temperature for 3 h, saturated sodium bicarbonate solution is slowly added dropwise to neutralize the mixture to make pH=8 to obtain a neutralized solution. The neutralize solution is extracted for three times with DCM solution (300 mL) to obtain organic phases. The organic phases are merged, dried with anhydrous Na₂SO₄, and concentrated under reduced pressure to obtain an intermediate amine (i.e., a fourth group-removed compound).

The intermediate amine is dissolved in anhydrous DCM (500 mL), and HATU (15.2 g, 40.0 mmol) and the compound 18 (11.6 g, 26 mmol) are sequentially added, after cooling to the temperature of 0° C., DIPEA (8.3 mL, 50.0 mmol) is slowly added dropwise to react for 30 min to obtain a mixture. After heating of the mixture to the room temperature for reacting for 14 h, an organic phase of the mixture is sequentially washed by diluted hydrochloric acid (300 mL) with a concentration of 2 M, washed by saturated sodium bicarbonate solution (300 mL), dried with anhydrous Na₂SO₄, and concentrated under reduced pressure to obtain a crude product. PE and EA with a ratio of 2:1 are used as an eluent to perform a flash column chromatography on the crude product to obtain the compound 28 (18.5 g, a total yield of 80%).

The compound 28 is detected through ¹HNMR, ¹³CNMR and HRMS, the compound 28 is a pure compound, and performance indicators or characterization data of the compound 28 are as follows: ¹H NMR(400 MHZ, CDCl₃) δ 7.64(d, J=15.5 Hz, 1H), 7.39(s, 1H), 7.10(d, J=7.3 Hz, 1H), 6.83(d, J=8.6 Hz, 2H), 6.74(t, J=11.7 Hz, 3H), 6.51(d, J=10.0 Hz, 1H), 6.00(ddd, J=22.4, 10.5, 5.3 Hz, 1H), 5.47(dd, J=8.6, 6.1 Hz, 2H), 5.36(dd, J=17.3, 1.4 Hz, 1H), 5.24(dd, J=10.5, 1.2 Hz, 1H), 4.90(d, J=10.0 Hz, 1H), 4.83(t, J=9.5 Hz, 2H), 4.48-4.44(m, 2H), 4.35(t, J=8.3 Hz, 1H), 3.82(dd, J=14.3, 8.0 Hz, 1H), 3.24(dd, J=13.7, 4.5 Hz, 1H), 3.10(dd, J=13.8, 7.5 Hz, 1H), 1.52-1.42(m, 3H), 1.39(s, 9H), 1.25(d, J=11.9 Hz, 9H), 1.11(dd, J=6.3, 3.5 Hz, 4H), 0.91(d, J=6.6 Hz, 3H), 0.82(t, J=7.2 Hz, 3H), 0.71(t, J=7.1 Hz, 3H); ¹³C NMR(100 MHz, CDCl₃) δ 176.26, 171.60, 170.19, 167.93, 165.15, 157.59, 155.97, 150.51, 137.82, 133.14, 130.60, 127.40, 122.80, 118.97, 117.53, 114.63, 94.99, 80.00, 74.11, 72.65, 68.65, 54.13, 52.54, 40.74, 38.95, 36.36, 33.35, 28.18, 27.01, 25.06, 18.70, 15.58, 13.59, 10.89; HR-ESIMS m/z: calculated for C₄₁H₅₇C₁₃N₄O₉SNa⁺[M+Na]⁺: 909.2912, found 909.2915.

(12) A synthesis process of a compound 29 is as follows:

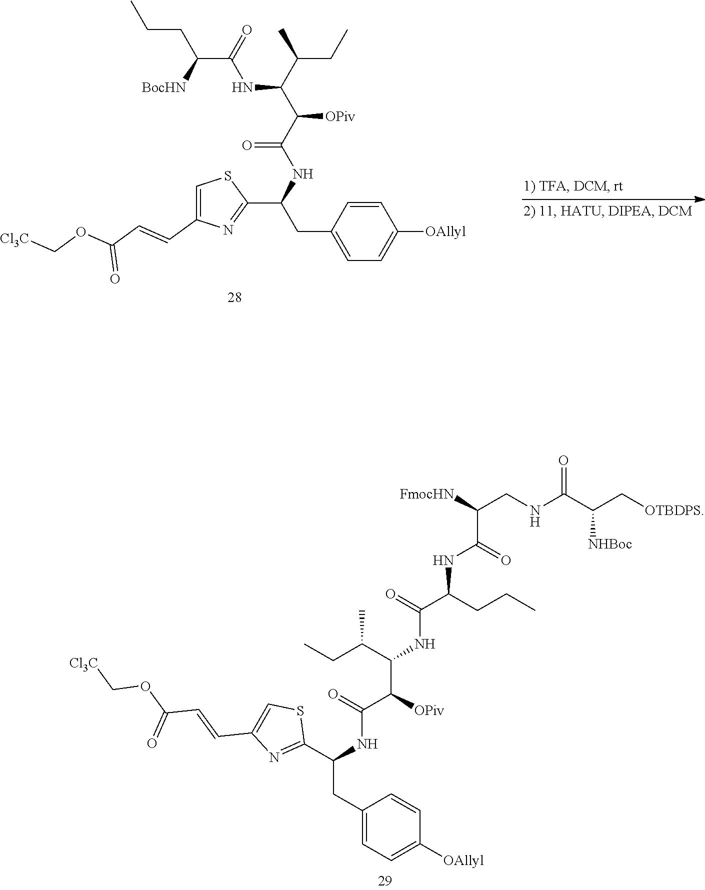

The compound 28 (17.8 g, 20.0 mmol) is dissolved in DCM solution (200 mL), and trifluoroacetic acid (30 mL) is added to obtain a mixture. After stirring of the mixture for reacting at the room temperature for 3 h, saturated sodium bicarbonate solution is slowly added dropwise to neutralize the mixture to make pH=8 to obtain a neutralized solution. The neutralize solution is extracted for three times with DCM solution (300 mL) to obtain organic phases. The organic phases are merged, dried with anhydrous Na₂SO₄, and concentrated under reduced pressure to obtain an intermediate amine (i.e., a fifth group-removed compound).

The intermediate amine is dissolved in anhydrous DCM (500 mL), and HATU (11.4 g, 30.0 mmol) and the compound 11 (15.0 g, 20.0 mmol) are sequentially added, after cooling to the temperature of 0° C., DIPEA (6.6 mL, 40.0 mmol) is slowly added dropwise to react for 30 min to obtain a mixture. After heating of the mixture to the room temperature for reacting for 14 h, an organic phase of the mixture is sequentially washed by diluted hydrochloric acid (300 mL) with a concentration of 2 M, washed by saturated sodium bicarbonate solution (300 mL), dried with anhydrous Na₂SO₄, and concentrated under reduced pressure to obtain a crude product. PE and EA with a ratio of 1:1 are used as an eluent to perform a flash column chromatography on the crude product to obtain the compound 29 (21.3 g, a total yield of 70%).

The compound 29 is detected through ¹HNMR, ¹³CNMR and HRMS, the compound 29 is a pure compound, and performance indicators or characterization data of the compound 29 are as follows: ¹H NMR(500 MHz, CDCl₃) δ 7.93-7.85(m, 3H), 7.78-7.63(m, 6H), 7.61-7.43(m, 6H), 7.46-7.36(m, 6H), 7.14-7.06(m, 3H), 6.80-6.70(m, 4H), 6.30(d, J=15.0 Hz, 1H), 6.24(s, 2H), 6.16-6.00(m, 2H), 5.67-5.54(m, 3H), 5.34(ddd, J=16.7, 13.8, 1.9 Hz, 1H), 5.16(t, J=7.0 Hz, 1H), 4.83(dd, J=12.4, 9.9 Hz, 1H), 4.77-4.67(m, 4H), 4.42(dd, J=12.5, 7.0 Hz, 1H), 4.37-4.22(m, 3H), 4.17(dd, J=12.4, 7.1 Hz, 1H), 3.81(d, J=12.3 Hz, 1H), 3.43(dd, J=12.5, 7.0 Hz, 1H), 3.29(ddt, J=12.5, 7.0, 1.1 Hz, 1H), 3.10(ddt, J=12.5, 6.9, 1.1 Hz, 1H), 2.69(tdd, J=12.9, 3.5, 2.2 Hz, 1H), 2.08-1.96(m, 1H), 1.94-1.82(m, 1H), 1.44(s, 9H), 1.08(d, J=20.0 Hz, 18H), 0.96-0.87(m, 6H), 0.81(t, J=8.0 Hz, 3H), 0.37(dqd, J=12.2, 7.9, 1.7 Hz, 1H); ¹³C NMR(125 MHz, CDCl₃) δ 177.23, 172.54, 172.02, 170.28, 170.13, 169.33, 168.82, 158.44, 157.94, 156.03, 150.72, 143.56, 141.18, 134.46, 133.80, 133.76, 132.73, 131.05, 130.57, 129.97, 128.81, 127.07, 126.38, 125.48, 123.07, 121.08, 117.83, 117.27, 115.06, 95.14, 79.69, 74.61, 73.88, 67.72, 66.72, 64.70, 54.73, 54.68, 54.31, 54.03, 52.98, 47.37, 40.72, 38.98, 34.38, 34.30, 28.34, 27.10, 26.83, 26.35, 15.29, 13.18, 11.50; HR-ESIMS m/z: calculated for C₇₈H₉₆C₁₃NO₁₄SSiNa⁺[M+Na]⁺: 1542.5571, found 1542.5575.

(13) A synthesis process of a key intermediate compound 2 is as follows:

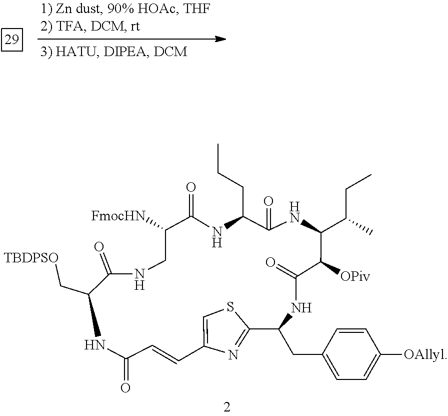

The compound 29 (10.0 g, 6.6 mmol) is dissolved in THF (200 mL), and acetic acid (50 mL) with a concentration of 90% and zinc dust (8.6 g, 132 mmol) are added to obtain a mixture. After stirring of the mixture for reacting at the room temperature for 2 h, the mixture is concentrated under reduce pressure to remove THF therein, and diluted by adding water (500 mL) to obtain a diluted solution. Saturated sodium bicarbonate solution is slowly added dropwise to neutralize the diluted solution to make pH=8 to obtain a neutralized solution. The neutralize solution is extracted for three times with DCM solution (500 mL) to obtain an extracted solution. The extracted solution is filtered to remove zinc dust therein to obtain a filtrate. The filtrate is separated into an organic phase and an aqueous phase. The aqueous phase is extracted for twice with DCM solution (400 mL) to obtain organic phases. The organic phases are merged, dried with anhydrous Na₂SO₄, and concentrated under reduced pressure to obtain an intermediate acid (i.e., a second hydrolyzed compound).

The intermediate acid is dissolved in DCM solution (200 mL), and trifluoroacetic acid (30 mL) is added to obtain a mixture. After stirring of the mixture for reacting at the room temperature for 3 h, the mixture is concentrated under reduced pressure to obtain an intermediate amine (i.e., a sixth group-removed compound).

The intermediate amine is dissolved in DCM solution (3000 mL), and HATU (7.5 g, 19.8 mmol) and DIPEA (5.0 mL, 30 mmol) are added to obtain a mixture. After stirring of the mixture for reacting at the room temperature for 1 day, the mixture is concentrated under reduced pressure to obtain a crude product. PE and EA with a ratio of 1:1 are used as an eluent to perform a flash column chromatography on the crude product to obtain the compound 2 (6.7 g, a total yield of 80%).

The compound 2 is detected through ¹HNMR, ¹³CNMR and HRMS, the compound 2 is a pure compound, and performance indicators or characterization data of the compound 2 are as follows: ¹H NMR(500 MHz, CDCl₃) δ 9.45(s, 1H), 9.32(s, 1H), 8.30(s, 1H), 7.82-7.74(m, 3H), 7.67(ddq, J=6.8, 4.5, 2.7, 2.3 Hz, 4H), 7.61(s, 1H), 7.56-7.48(m, 3H), 7.48-7.39(m, 2H), 7.42-7.36(m, 6H), 7.09(dt, J=7.6, 1.1 Hz, 2H), 6.82-6.76(m, 2H), 6.67(s, 1H), 6.60(d, J=15.0 Hz, 1H), 6.46(d, J=15.0 Hz, 1H), 6.24(s, 1H), 6.16-6.00(m, 2H), 5.69(t, J=7.0 Hz, 1H), 5.59(ddd, J=13.8, 10.0, 1.7 Hz, 1H), 5.41(s, 1H), 5.34(ddd, J=16.9, 13.8, 1.8 Hz, 1H), 5.16(t, J=7.0 Hz, 1H), 4.81-4.68(m, 5H), 4.53(t, J=7.0 Hz, 1H), 4.46-4.32(m, 2H), 4.17(dd, J=12.5, 7.0 Hz, 1H), 4.08(dd, J=12.6, 7.1 Hz, 1H), 3.62(dd, J=12.5, 7.0 Hz, 1H), 3.35(ddt, J=12.4, 7.1, 1.0 Hz, 1H), 3.09(ddt, J=12.5, 7.0, 1.1 Hz, 1H), 2.07-1.89(m, 2H), 1.81(tt, J=12.6, 2.1 Hz, 1H), 1.30-1.15(m, 2H), 1.08(d, J=20.0 Hz, 19H), 0.92(dd, J=9.1, 7.3 Hz, 6H), 0.85(t, J=8.0 Hz, 3H); ¹³C NMR(125 MHz, CDCl₃) δ 177.23, 172.54, 172.02, 170.28, 170.13, 166.76, 158.44, 157.94, 150.10, 143.56, 141.18, 134.46, 133.76, 133.39, 132.73, 131.05, 130.57, 129.97, 128.81, 127.07, 126.38, 125.48, 123.07, 121.08, 119.85, 117.83, 115.06, 74.61, 67.72, 66.72, 64.70, 54.68, 54.40, 54.31, 54.03, 52.98, 47.37, 40.72, 38.98, 34.38, 34.30, 27.10, 26.83, 26.35, 15.29, 13.18, 11.50; HR-ESIMS m/z: calculated for C₇₁H₈₅N₇O₁₁SSiNa⁺[M+Na]⁺: 1294.5797, found 1294.5801.

(14) A synthesis process of a compound 31 is as follows:

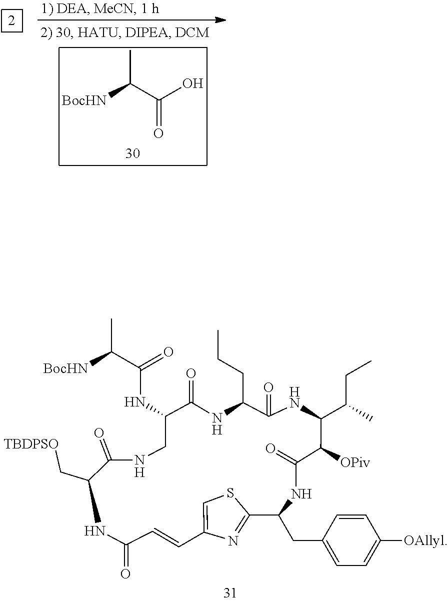

The compound 2 (6.0 g, 4.7 mmol) is dissolved in acetonitrile (200 mL), and diethylamine (20 mL) is added to obtain a mixture. After stirring of the mixture for reacting at the room temperature for 2 h, the mixture is concentrated under reduced pressure and dried in vacuum to obtain an intermediate amine.

The intermediate amine is dissolved in anhydrous DCM (200 mL), and HATU (3.8 g, 10.0 mmol) and a compound 30 (0.95 g, 5.0 mmol) are sequentially added, after cooling to the temperature of 0° C., DIPEA (2.5 mL, 15.0 mmol) is slowly added dropwise to react for 30 min to obtain a mixture. After heating of the mixture to the room temperature for reacting for 15 h, an organic phase of the mixture is sequentially washed by diluted hydrochloric acid (300 mL) with a concentration of 2 M, washed by saturated sodium bicarbonate solution (300 mL), dried with anhydrous Na₂SO₄, and concentrated under reduced pressure to obtain a crude product. Pure EA is used as an eluent to perform a flash column chromatography on the crude product to obtain the compound 31 (4.6 g, a total yield of 80%).

The compound 31 is detected through ¹HNMR, ¹³CNMR and HRMS, the compound 31 is a pure compound, and performance indicators or characterization data of the compound 31 are as follows: ¹H NMR(500 MHz, CDCl₃) δ 7.67(ddq, J=6.7, 4.5, 2.7, 2.3 Hz, 4H), 7.55-7.47(m, 3H), 7.45-7.36(m, 7H), 7.09-7.04(m, 2H), 6.83-6.77(m, 2H), 6.46(t, J=7.5 Hz, 2H), 6.28(s, 1H), 6.24(s, 1H), 6.13(d, J=0.1 Hz, 1H), 6.07(s, 1H), 5.64-5.54(m, 2H), 5.17(q, J=6.7 Hz, 2H), 4.82-4.71(m, 2H), 4.56(dt, J=14.0, 6.2 Hz, 2H), 4.51-4.38(m, 2H), 4.17(dd, J=12.3, 6.8 Hz, 1H), 3.67(dd, J=12.5, 7.0 Hz, 1H), 3.30(ddt, J=12.5, 7.0, 1.1 Hz, 1H), 3.20(dd, J=12.5, 7.0 Hz, 1H), 3.10(ddt, J=12.3, 7.0, 1.1 Hz, 1H), 2.02(dddd, J=13.7, 8.5, 6.9, 3.3 Hz, 1H), 1.87-1.72(m, 3H), 1.44(s, 9H), 1.42-1.27(m, 4H), 1.08(d, J=20.0 Hz, 18H), 1.02-0.84(m, 10H); ¹³C NMR(125 MHz, CDCl₃) δ 177.23, 173.56, 172.54, 172.02, 170.28, 170.13, 166.76, 157.94, 155.50, 150.10, 134.46, 133.76, 133.39, 132.73, 131.05, 130.57, 129.97, 128.81, 123.07, 119.85, 117.83, 115.06, 79.64, 74.61, 67.72, 64.70, 54.40, 54.31, 54.03, 52.98, 50.85, 50.15, 40.72, 38.98, 34.38, 34.30, 28.34, 27.10, 26.83, 26.35, 19.44, 17.99, 15.29, 13.18, 11.50; HR-ESIMS m/z: calculated for C₆₄H₈₈N₈O₁₂SSNa⁺[M+Na]⁺: 1243.6012, found 1243.6015.

(15) A synthesis process of a compound 33 is as follows:

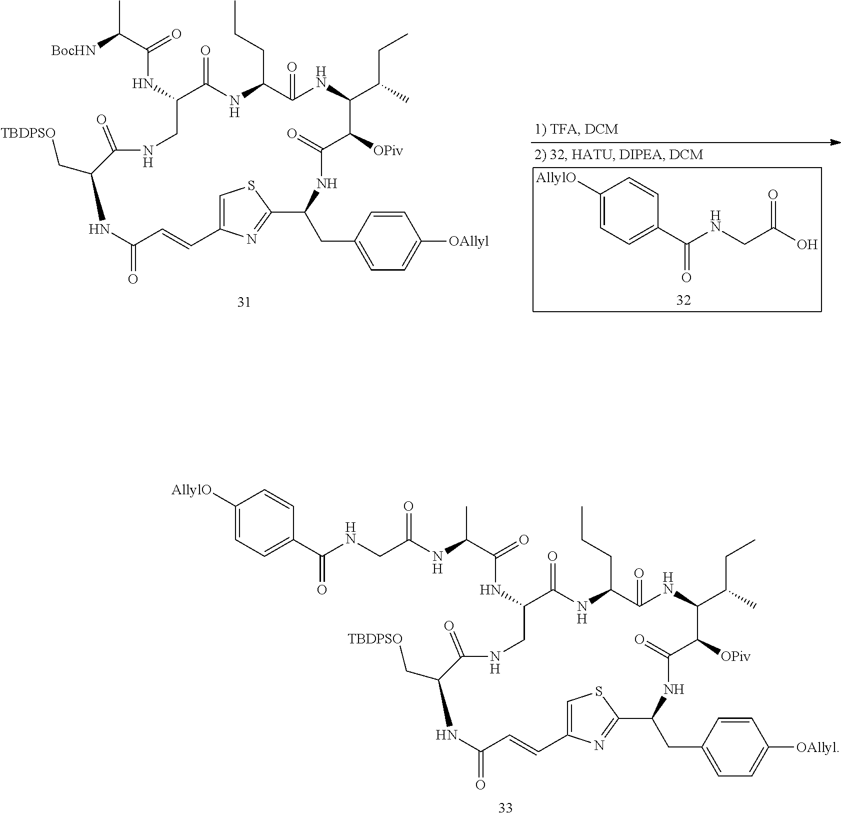

The compound 31 (4.6 g, 3.8 mmol) is dissolved in DCM solution (100 mL), and trifluoroacetic acid (15 mL) is added to obtain a mixture. After stirring of the mixture for reacting at the room temperature for 3 h, the mixture is concentrated under reduced pressure and dried in the vacuum to obtain an intermediate amine.

The intermediate amine is dissolved in anhydrous DCM (100 mL), and HATU (3.0 g, 8.0 mmol) and a compound 32 (1.0 g, 4.3 mmol) are sequentially added, after cooling to the temperature of 0° C., DIPEA (2.5 mL, 15.0 mmol) is slowly added dropwise to react for 30 min to obtain a mixture. After heating of the mixture to the room temperature for reacting for 15 h, an organic phase of the mixture is sequentially washed by diluted hydrochloric acid (300 mL) with a concentration of 2 M, washed by saturated sodium bicarbonate solution (300 mL), dried with anhydrous Na₂SO₄, and concentrated under reduced pressure to obtain a crude product. MeOH and EA with a ratio of 1:10 are used as an eluent to perform a flash column chromatography on the crude product to obtain the compound 33 (3.3 g, a total yield of 70%).

The compound 33 is detected through ¹HNMR, ¹³CNMR and HRMS, the compound 33 is a pure compound, and performance indicators or characterization data of the compound 33 are as follows: ¹H NMR(500 MHz, CDCl₃) δ 8.70(s, 1H), 8.20(s, 1H), 8.00(s, 1H), 7.83(s, 1H), 7.67(ddq, J=6.8, 4.5, 2.8, 2.2 Hz, 4H), 7.58-7.52(m, 2H), 7.50(s, 5H), 7.42-7.36(m, 6H), 7.33(d, J=15.0 Hz, 1H), 7.06(dt, J=7.6, 1.2 Hz, 2H), 6.96-6.90(m, 2H), 6.79-6.73(m, 2H), 6.46(d, J=15.0 Hz, 1H), 6.16-6.00(m, 3H), 5.59(ddt, J=9.9, 2.1, 1.1 Hz, 2H), 5.34(ddd, J=16.0, 2.3, 1.2 Hz, 2H), 5.26(t, J=0.0 Hz, 1H), 5.16(t, J=6.9 Hz, 1H), 4.67(dt, J=6.2, 1.1 Hz, 4H), 4.54(t, J=7.0 Hz, 1H), 4.47-4.38(m, 2H), 4.29(t, J=7.0 Hz, 1H), 4.16(qd, J=6.9, 4.6 Hz, 2H), 3.83(dd, J=12.4, 7.1 Hz, 1H), 3.75(d, J=15.6 Hz, 2H), 3.58(dd, J=12.4, 7.1 Hz, 1H), 3.23(ddt, J=12.2, 7.0, 0.9 Hz, 1H), 2.98(ddt, J=12.2, 6.9, 1.0 Hz, 1H), 2.02(tp, J=6.9, 3.3 Hz, 2H), 1.72-1.61(m, 1H), 1.60-1.45(m, 3H), 1.12-1.04(m, 21H), 1.05-0.95(m, 1H), 0.96-0.89(m, 6H), 0.90(d, J=7.8 Hz, 2H); ¹³C NMR(125 MHz, CDCl₃) δ 177.23, 174.62, 172.54, 172.02, 170.13, 169.97, 169.33, 168.43, 166.76, 160.36, 157.94, 150.10, 134.46, 133.76, 133.39, 132.73, 131.05, 130.57, 130.49, 129.97, 129.48, 128.81, 123.07, 119.85, 117.83, 115.06, 114.64, 74.61, 67.72, 64.70, 54.40, 54.31, 54.03, 52.98, 50.85, 49.44, 43.21, 40.72, 39.14, 38.98, 34.38, 34.30, 27.10, 26.83, 26.35, 19.44, 17.10, 15.29, 13.18, 11.50; HR-ESIMS m/z: calculated for C₇₁H₉₁N₉O₁₃SSiNa⁺[M+Na]⁺: 1360.6226, found 1360.6228.

(16) A synthesis process of the CyclotheonellazoleA (i.e., the compound 1) is as follows:

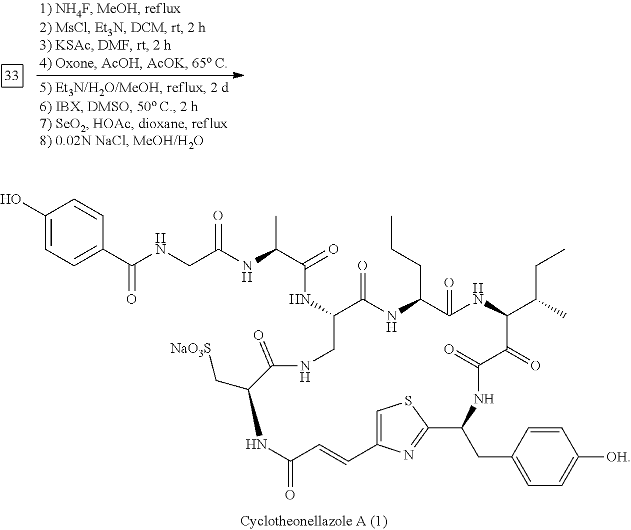

The compound 33 (3.3 g, 2.6 mmol) is dissolved in MeOH solution (200 mL), and NH₄F solid (4.8 g, 130 mmol) is added to obtain a mixture. After heating and refluxing of the mixture for reacting for 12 h, the mixture is concentrated under reduced pressure to remove MeOH therein to obtain a residue, the residue is dissolved in EA (600 mL) to obtain a residue solution. The residue solution is washed by saturated sodium bicarbonate solution (150 mL), dried with anhydrous Na₂SO₄, and concentrated under reduced pressure to obtain a first intermediate (i.e., a third group-removed compound).

The first intermediate obtained in the previous step is dissolved in anhydrous DCM (200 mL), and Et₃N (0.75 mL, 5.3 mmol) is added, after cooling to the temperature of 0° C., methanesulfonyl chloride (MsCl) (0.3 mL, 4 mmol) is slowly added dropwise to react for 30 min to obtain a mixture. After heating of the mixture to the room temperature for reacting for 2 h, the mixture is diluted with DCM solution (400 mL) to obtain a diluted solution. The diluted solution is sequentially washed by diluted hydrochloric acid (100 mL) with a concentration of 1 M, washed by saturated sodium bicarbonate solution (100 mL), dried with anhydrous Na₂SO₄, and concentrated under reduced pressure to obtain a second intermediate (i.e., activated compound).

The second intermediate obtained in the previous step is dissolved in DMF (20 mL), and potassium thioacetate (3.0 g, 26.5 mmol) is added to obtain a mixture. After stirring of the mixture for reacting at the room temperature for 4 h, the mixture is quenched by adding water (500 mL), and extracted for twice with EA (400 mL) to obtain organic phases. The organic phases are merged, washed by a saturated sodium chloride aqueous solution (300 mL), dried with anhydrous Na₂SO₄, and concentrated under reduced pressure to obtain a third intermediate (thioester).

The third intermediate obtained in the previous step is dissolved in acetic acid solution (100 mL), and potassium acetate (32.5 g, 330 mmol) and potassium monopersulphate triple salt (Oxone) (32.6 g, 53 mmol) are added to obtain a mixture. After heating of the mixture to a temperature of 65° C. for reacting for 14 h, the mixture is cooled to the temperature of 0° C., and the mixture is quenched by adding water (500 mL) to obtain a quenched solution. A sodium hydroxide solid is slowly added to neutralize the quenched solution to make pH=8 to obtain a neutralized solution. The neutralize solution is extracted for three times with EA (300 mL) to obtain organic phases. The organic phases are merged, washed by saturated sodium chloride aqueous solution (200 mL), dried with anhydrous Na₂SO₄, and concentrated under reduced pressure to obtain an intermediate sulfonic acid (i.e., a first sulfurous acid).

The intermediate sulfonic acid obtained in the previous step is dissolved in MeOH solution (150 mL), and Et₃N (15 mL) and water (15 mL) are added to obtain a mixture. After heating and refluxing of the mixture for reacting for 2 days, the mixture is concentrated under reduced pressure to remove all solvents therein, and fully dried to obtain a fourth intermediate (i.e., secondary alcohol).

The fourth intermediate obtained in the previous step is dissolved in DMSO (30 mL), and IBX (2.3 g, 8.0 mmol) is added to obtain a mixture. After heating of the mixture to a temperature of 60° C. for reacting for 2 h, the mixture is quenched by adding water (400 mL), and extracted for three times with EA (300 mL) to obtain organic phases. The organic phases are merged, washed by saturated sodium chloride aqueous solution (200 mL), dried with anhydrous Na₂SO₄, and concentrated under reduced pressure to obtain a fifth intermediate (i.e., α-ketoamide).

The fifth intermediated obtained in the previous step is dissolved in 1,4-dioxane (100 mL), and acetic acid (0.75 mL, 13.3 mmol) and selenium dioxide (0.9 g, 8.0 mmol) are added to obtain a mixture. After heating and refluxing of the mixture for reacting for 4 h, the mixture is concentrated under reduced pressure to obtain a crude product. A column chromatography is performed on the crude product with silica gel to obtain a crude product intermediate.

The crude product intermediate obtained in the previous step is dissolved in a mixed solvent with MeOH and water (1:1, 10 mL), and sodium chloride solution (1 M, 2.5 mL) is added to obtain a mixture. After stirring of the mixture for reacting at the room temperature for 1 h, the mixture is concentrated under reduced pressure, and purified by using an inverted high-performance liquid chromatography (HPLC) to obtain a pure compound 1 (0.93 g, a total yield of 35%).

The compound 1 is detected through ¹HNMR, ¹³CNMR and HRMS, the compound 1 is a pure compound, and performance indicators or characterization data of the compound 1 are as follows: ¹H NMR(400 MHZ, d₆-DMSO) δ 10.01(s, 1H), 9.44(d, J=7.9 Hz, 1H), 9.32(s, 1H), 8.62(d, J=7.0 Hz, 1H), 8.50(t, J=5.8 Hz, 1H), 8.36(d, J=9.4 Hz, 1H), 8.16(d, J=9.3 Hz, 1H), 8.04(d, J=7.8 Hz, 1H), 7.98(d, J=7.6 Hz, 1H), 7.87(s, 1H), 7.87(brs, 1H), 7.74(d, J=8.7 Hz, 2H), 7.41(d, J=15.1 Hz, 1H), 7.18(d, J=8.5 Hz, 2H), 6.80(d, J=8.7 Hz, 2H), 6.76(d, J=15.2 Hz, 1H), 6.70(d, J=8.5 Hz, 2H), 5.22(m, 1H), 5.05(dd, J=9.5, 3.4 Hz, 1H), 4.75(m, 1H), 4.33(m, 1H), 4.32(m, 1H), 4.04(m, 1H), 3.90(dd, J=16.5, 6.0 Hz, 1H), 3.79(dd, J=16.5, 5.6 Hz, 1H), 3.74(m, 1H), 3.27(m, 1H), 3.10(dd, J=13.7, 5.7 Hz, 1H), 3.03(dd, J=13.7, 5.7 Hz, 1H), 2.72(dd, J=13.5, 5.3 Hz, 1H), 2.49(m, 1H), 2.42(m, 1H), 2.30(m, 1H), 1.63(m, 1H), 1.56(m, 1H), 1.33(m, 1H), 1.17(d, J=7.0 Hz, 3H), 1.15(m, 1H), 1.08(m, 1H), 0.93(t ,J=7.2 Hz, 3H), 0.78(d, J=6.9 Hz, 3H), 0.73(t, J=7.4 Hz, 3H); ¹³C NMR(100 MHz, d₆-DMSO) δ 198.0, 174.8, 171.4, 171.1, 170.67, 170.57, 168.8, 166.3, 164.9, 163.7, 160.3, 156.2, 149.5, 132.6, 130.4, 129.3, 127.3, 124.6, 123.3, 123.2, 115.2, 114.8, 60.1, 54.4, 54.3, 50.9, 50.5, 49.3, 47.8, 42.6, 40.8, 39.2, 36.9, 32.2, 23.3, 19.7, 18.4, 16.1, 13.8, 11.8; HR-ESIMS m/z: calculated for C₄₄H₅₄N₉NaO₁₄S₂Na⁺[M+Na]⁺: 1042.3129, found 1042.3131.

### Embodiment 2 An Inhabitation Effect of the CA (i.e., the Compound 1) to Transmembrane Serine Protease 2 (TMPRSS2)

1. Experimental materials include a TMPRSS2 detection kit (78083) of a BPS Bioscience company and CA.
2. Experimental methods are as follows. In a black 96-well plate, a blank well group, a negative control well group and a sample well group are set, and a detection system is 50 microliters (μL). The blank well group is added with 40 μL of buffer, the negative control well group is added with 30 μL of TMPRSS2 protease and 10 μL of buffer, the sample well group is added with 30 μL of TMPRSS2 protease and 10 μL of CA solution (respectively with concentrations of 0.1 micromole per liter abbreviated as μM, 1 μM, 10 μM, 30 μM, 100 μM, 150 μM, and 200 μM), and the three groups are incubated at the room temperature for 30 min. Each well is added with 10 μL of substrate, after mixing evenly, fluorescence value of each well is immediately detected at excitation/emission (Ex/Em)=383/455 nanometers (nm), every 30 seconds, and a dynamic process within a range of 0-40 min is observed. Two different time points (T1 and T2) are selected within a linear range of the dynamic process to obtain fluorescence values (RFUT1 and RFUT2) corresponding to the time points, a slope of the fluorescence values is calculated, and the slope can be regarded as an enzyme activity of each well. A formula of the slope is slope=(RFUT2−RFUT1)/(T2−T1). An enzyme activity of the negative control well group is taken as 100% to calculate a relative enzyme activity of each group relative to the negative control well group. A half inhibitory concentration (IC₅₀) is calculated through an Origin 2021 software.
3. Experimental result is as follows.

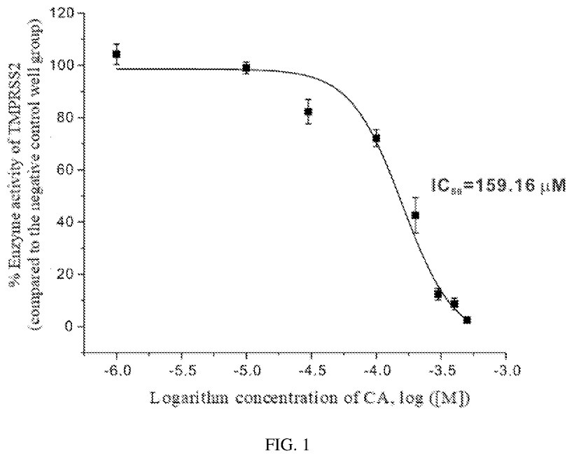

As shown in FIG. 1, the CA can inhabit an enzyme activity of the TMPRSS2 protease, and a value of IC₅₀ is 19.17 μM.

### Embodiment 3 An Inhabitation Effect of the CA (i.e., the Compound 1) to Mainprotease (Mpro) of SARS-COV-2 Virus

1. Experimental materials include a Mpro detection kit (78042-2) of the BPS Bioscience company and CA.
2. Experimental methods are as follows. In a black 384-well plate, a blank well group, a negative control well group and a sample well group are set, and a detection system is 25 μL. The blank well group is added with 12.5 μL of buffer, the negative control well group is added with 10 μL of Mpro and 2.5 μL of buffer, the sample well group is added with 10 μL of Mpro and 2.5 μL of CA solution (respectively with concentrations of 1 μM, 10 μM, 30 μM, 100 μM, 200 μM, 300 μM, 400 μM and 500 μM), and the three groups are incubated at the room temperature for 30 min. Each well is added with 12.5 μL of substrate, after mixing evenly, fluorescence value of each well is immediately detected at Ex/Em=360/460 nm, every 20 seconds, and a dynamic process within 4 h is observed. Two different time points (T1 and T2) are selected within a linear range of the dynamic process to obtain fluorescence values (RFUT1 and RFUT2) corresponding to the time points, a slope of the fluorescence values is calculated, and the slope can be regarded as an enzyme activity of each well. A formula of the slope is slope=(RFUT2−RFUT1)/(T2−T1). An enzyme activity of the negative control well group is taken as 100% to calculate a relative enzyme activity of each group relative to the negative control well group. A half inhibitory concentration (IC₅₀) is calculated through the Origin 2021 software.
3. Experimental result is as follows.

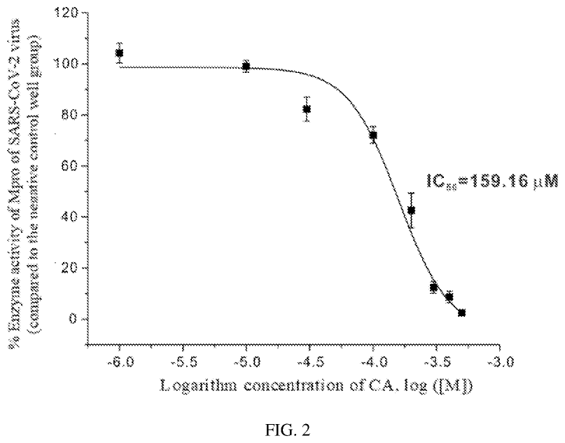

As shown in FIG. 2, the CA can inhabit an enzyme activity of the Mpro, and a value of IC₅₀ is 159.16 μM.

### Embodiment 4 An Inhabitation Effect of the CA (i.e., the Compound 1) to Neutrophil Clastase (NE)

1. Experimental materials include a NE protease detection kit (ab118971) of an Abcam company and CA.
2. Experimental methods are as follows. In a black 96-well plate, a blank well group, a negative control well group and a sample well group are set, and a detection system is 100 μL. The blank well group is added with 75 μL of buffer, the negative control well group is added with 50 μL of NE protease and 25 μL of buffer, the sample well group is added with 50 μL of NE protease and 25 μL of CA solution (respectively with concentrations of 0.00001 μM, 0.0001 μM, 0.001 μM, 0.003 μM, 0.01 μM and 0.1 μM), and the three groups are incubated at a temperature of 37° C. for 5 min. Each well is added with 25 μL of substrate, after mixing evenly, fluorescence value of each well is immediately detected at Ex/Em=400/505 nm, every 20 seconds, and a dynamic process within a range of 0-40 min is observed. Two different time points (T1 and T2) are selected within a linear range of the dynamic process to obtain fluorescence values (RFUT1 and RFUT2) corresponding to the time points, a slope of the fluorescence values is calculated, and the slope can be regarded as an enzyme activity of each well. A formula of the slope is slope=(RFUT2−RFUT1)/(T2−T1). An enzyme activity of the negative control well group is taken as 100% to calculate a relative enzyme activity of each group relative to the negative control well group. A half inhibitory concentration (IC₅₀) is calculated through the Origin 2021 software.
3. Experimental result is as follows.

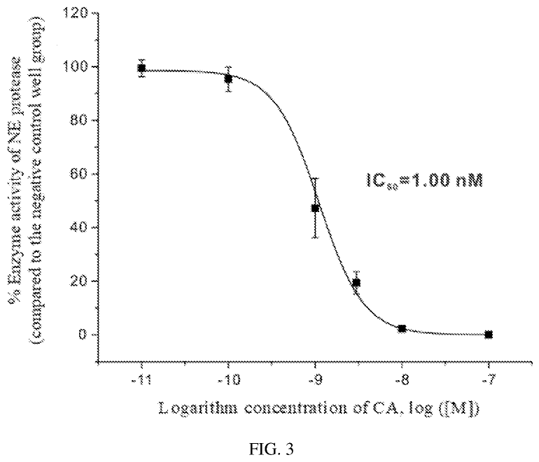

As shown in FIG. 3, the CA can inhabit an enzyme activity of the NE protease, and a value of IC₅₀ is 1.00 nanomolar per liter (nM).

The Cyclotheonellazole A natural product is successfully synthesized in the disclosure by using a classical retrosynthesis analysis, a stereoscopic structure of the Cyclotheonellazole A natural product is confirmed, extremely excellent protease inhibitory activity of the Cyclotheonellazole A natural product is confirmed by combining a protease inhibition activity experiment, and the Cyclotheonellazole A has a strong application prospect in a pharmaceutical industry.

The above are merely embodiments of the disclosure and are not intended to limit it, any modifications, equivalent substitutions and improvements made within a spirit and principles of the disclosure shall be included in a scope of protection of the disclosure.

What is claimed is:

1. A compound or a pharmaceutically acceptable salt thereof, the compound having a structure represented by a general formula (I) expressed as follows: 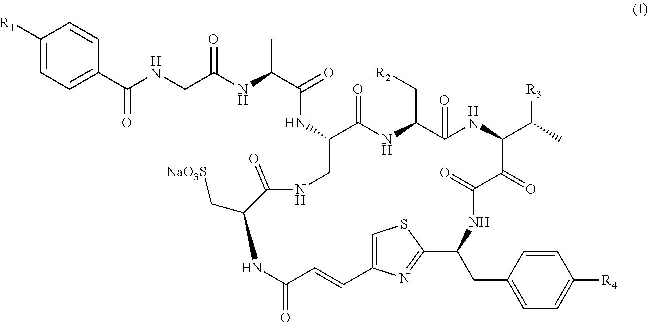
wherein R₁, R₂, R₃ and R₄ are independently selected form the group consisting of a hydrogen group (H), an alkyl group with a carbon atom in a range of 1-6, an alkoxy group with a carbon atom in a range of 1-6, a halogen group, a hydroxyl group, an amino group, a nitro group, a cyano group and a sulfydryl group.

2. The compound or the pharmaceutically acceptable salt thereof as claimed in claim 1, wherein the R₁ is the hydroxyl group, the R₂ is the alkyl group with the carbon atom in a range of 1-3, the R₃ is the alkyl group with the carbon atom in a range of 1-3, and the R₄ is the hydroxyl group.

3. The compound or the pharmaceutically acceptable salt thereof as claimed in claim 2, wherein the compound has a structure represented by a formula (la) expressed as follows:

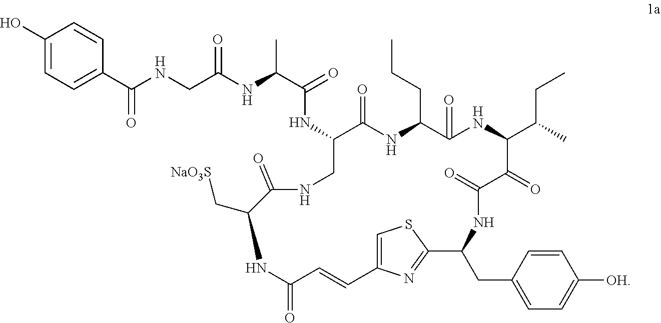

4. A synthetic method of a CyclotheonellazoleA, comprising:
removing a 9-fluorenylmethyloxycarbonyl (Fmoc) protection group from a compound 2 with diethylamine (DEA) to obtain a first group-removed compound, and performing a peptide grafting reaction on the first group-removed compound and a compound 30 to obtain a compound 31; and removing a t-butyloxy carbonyl (Boc) protection group from the compound 31 with trifluoroacetic acid (TFA) to obtain a second group-removed compound, and performing a peptide grafting reaction on the second group-removed compound and a compound 32 to obtain a compound 33; wherein a specific synthesis process of the compound 33 is as follows: [symbol]
;
removing a tert-butyldiphenylsilyl (TBDPS) protection group from the compound 33 with ammonium fluoride (NH₄F) to obtain a third group-removed compound, and activating a hydroxyl group of the third group-removed compound with methanesulfonyl chloride (MsCl) to obtain activated compound; performing a bimolecular nucleophilic substitution (SN₂) reaction on the activated compound with potassium thioacetate (KSAc) to obtain a thioester; oxidizing the thioester with potassium monopersulphate triple salt (Oxone) to obtain a first sulfurous acid; removing a pivaloyl (Piv) group from the first sulfurous acid with triethylamine (C₆H₁₅N) in a reflux condition of methanol (MeOH) to obtain a secondary alcohol; oxidizing the secondary alcohol with 2-iodoxybenzoic acid (IBX) to obtain a α-ketoamide; and oxidizing the α-ketoamide with selenium dioxide (SeO₂) to remove two allyl groups from the α-ketoamide, to thereby obtain a second sulfurous acid, and adding a saturated sodium chloride solution into the second sulfurous acid to obtain the CyclotheonellazoleA; wherein a specific synthesis process of the CyclotheonellazoleA is as follows: 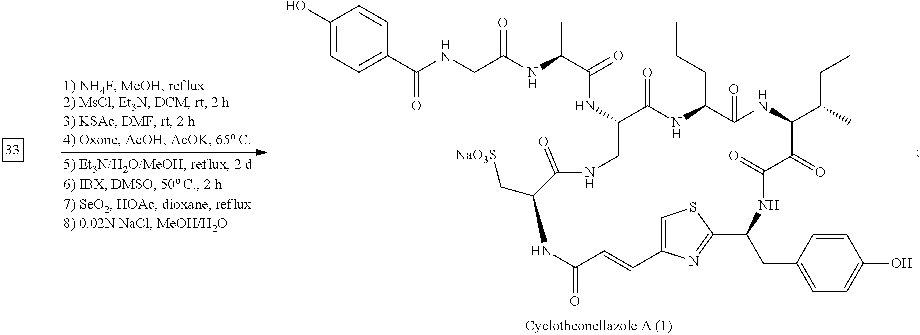
and
wherein structures of the compound 2, the compound 30 and the compound 33 are as follows: [symbol]
; [symbol]
; [symbol]
.

5. The synthesis method as claimed in claim 4, wherein a synthesis method of the compound 2 comprises:
performing a condensation reaction on a compound 19 and a compound 20 to obtain a compound 21, protecting a primary alcohol group of the compound 21 with tert-butyldimethylsilyl chloride (TBSCL) to obtain a compound 22, and performing a thionation reaction on the compound 22 with a Lawesson's reagent to obtain a compound 23; removing a t-butyldimethylsilyl (TBS) protection group from the compound 23 with the NH₄F to obtain a compound 24, performing a ring-closing reaction on the compound 24 with diethylaminosulfurtrifluoride (DAST) to obtain a ring-closed compound, oxidizing the ring-closed compound with 1,8-diazabicyclo[5.4.0]undec-7-ene (DBU) and bromotrichloro methane (CBrCl₃) to obtain a first oxidized compound, and adding an allyl group into the first oxidized compound to obtain a compound 25; reducing a methyl ester group of the compound 25 with sodium borohydride (NaBH₄) to obtain a reduced compound, oxidizing a primary alcohol group of the reduced compound with the IBX to obtain a second oxidized compound, and performing a Wittig reaction on the second oxidized compound to obtain a compound 26; and hydrolyzing an ethyl ester group of the compound 26 with sodium hydroxide (NaOH) to obtain a first hydrolyzed compound, and performing an esterification reaction on the first hydrolyzed compound and trichloroethanol (C₂H₃Cl₃₀) to obtain a compound 27; wherein a specific synthesis process of the compound 27 is as follows: 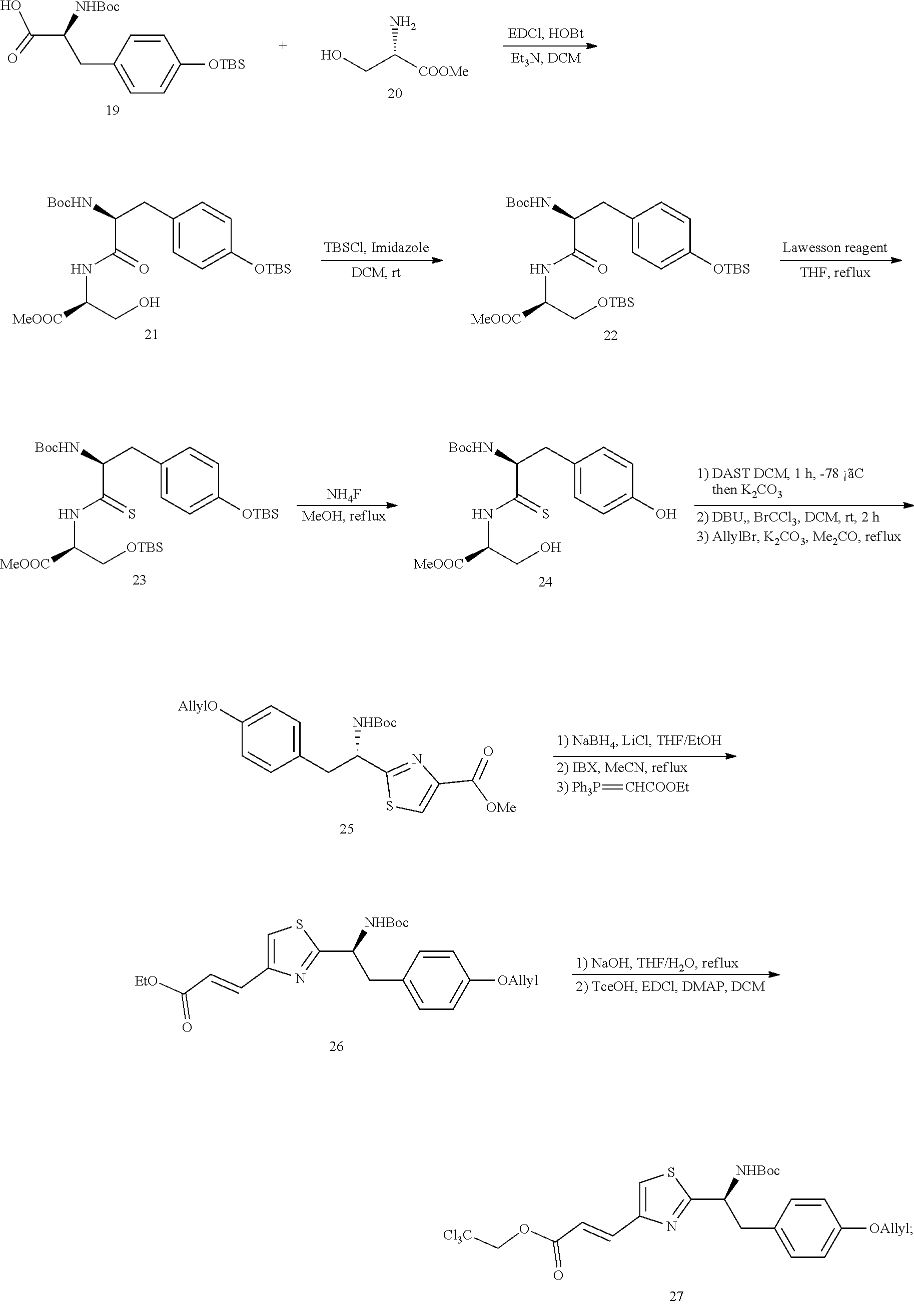
removing a Boc protection group from the compound 27 with the trifluoroacetic acid to obtain a fourth group-removed compound, and performing a peptide grafting reaction on the fourth group-removed compound and a compound 18 to obtain a compound 28; and removing a Boc protection group from the compound 28 with the trifluoroacetic acid to obtain a fifth group-removed compound, and performing a peptide grafting reaction on the fifth group-removed compound and a compound 11 to obtain a compound 29; wherein a specific synthesis process of the compound 29 is as follows: 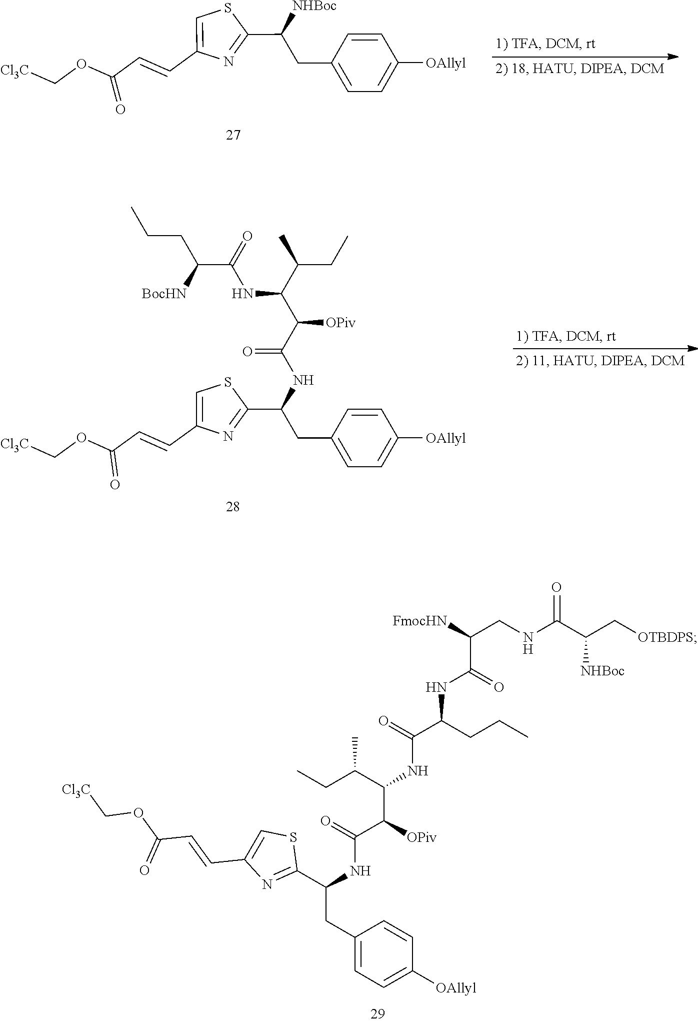
hydrolyzing a trichloroethyl ester group of the compound 29 in an acetic acid solution with zinc (Zn) dust to obtain a second hydrolyzed compound, removing a Boc protection group from the second hydrolyzed compound with the trifluoroacetic acid to obtain a sixth group-removed compound, and performing an intra-molecular macrocyclic-closing reaction on the sixth group-removed compound in a dichloromethane (DCM) solution with 2-(7-azabenzotriazol-1-yl)-N,N,N′,N′-tetramethyluronium hexafluorophosphate (HATU) to obtain the compound 2; wherein a specific synthesis process of the compound 2 is as follows: 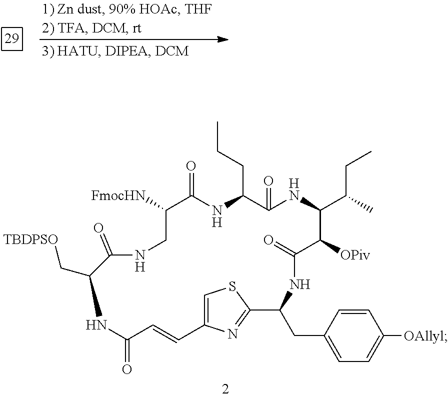
and
wherein structures of the compound 11 and the compound 18 are as follows: [symbol]
; [symbol] .

6. The synthesis method as claimed in claim 5, wherein a synthesis method of the compound 18 comprises:
taking a compound 12 as a starting material, performing a methyl esterification on the compound 12 to obtain a methyl esterified compound, protecting an amino group of the methyl esterified compound with di-tert-butyl dicarbonate to obtain a first intermediate compound, and reducing a methyl ester group of the first intermediate compound with the NaBH₄ to obtain a compound 13; oxidizing a primary alcohol group of the compound 13 with the IBX to obtain an aldehyde, performing a reaction between the aldehyde and acetone cyanohydrin to obtain a second intermediate compound, refluxing and hydrolyzing, by using a MeOH solution of hydrogen chloride, the second intermediate compound without purifying to obtain a third hydrolyzed compound, and protecting an amino group of the third hydrolyzed compound with the di-tert-butyl dicarbonate to obtain two diastereoisomers, a compound 14 and a compound 14′; wherein a specific synthesis process of the compound 14 and the compound 14′ is as follows: [symbol]
;
performing a reaction on the compound 14 and pivaloyl chloride (CsH₉ClO) to obtain a compound 15, removing a Boc protection group of the compound 15 with the trifluoroacetic acid to obtain a seventh group-removed compound, and preforming a peptide grafting reaction between the seventh group-removed compound and a compound 16 to obtain a compound 17; and hydrolyzing a methyl ester group of the compound 17 in a reflux condition of pyridine with lithium iodide to obtain the compound 18; wherein a specific synthesis process of the compound 18 is as follows: [symbol]
; and
wherein a structure of the compound 16 is as follows: [symbol]
.

7. The synthesis method as claimed in claim 5, wherein a synthesis method of the compound 11 comprises:
removing a Boc protection group of a compound 8 with the trifluoroacetic acid to obtain an eighth group-removed compound, and preforming a peptide grafting reaction on the eighth group-removed compound and a compound 9 to obtain a compound 10; and removing a phenoxyacetyl (Pac) protection group from the compound 10 in the acetic acid solution with the zinc dust to obtain the compound 11; wherein a specific synthesis process of the compound 11 is as follows: [symbol]
; and
wherein a structure of the compound 9 is as follows: [symbol]
.

8. The synthesis method as claimed in claim 7, wherein a synthesis method of the compound 8 comprises:
taking a compound 6 as a starting material, protecting an amino group of the compound 6 with N-(9-fluorenylmethoxycarbonyloxy)succinimide (Fmoc-Osu) to obtain a compound 7, and performing a Hofmann rearrangement reaction on the compound 7 to obtain a compound 3; and
protecting an amino group of the compound 3 with the di-tert-butyl dicarbonate (BOC₂O), and protecting a carboxyl group of the compound 3 with phenacyl bromide (PacBr), to obtain the compound 8; wherein a specific synthesis process of the compound 8 is as follows:

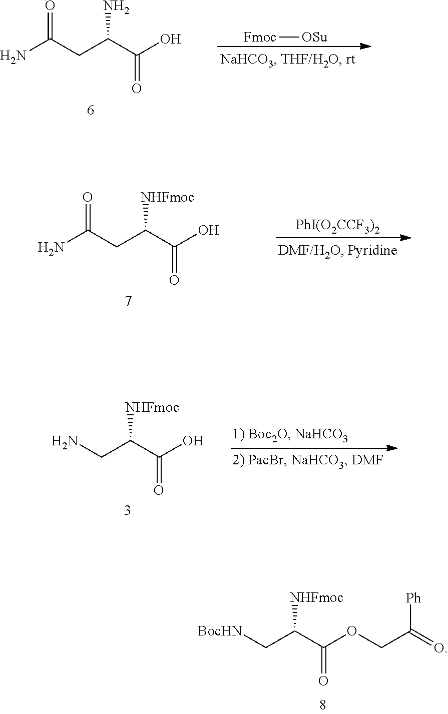

9. A pharmaceutical composition, comprising the compound or the pharmaceutically acceptable salt thereof as claimed in claim 1.

10. An application method of the compound or the pharmaceutically acceptable salt thereof as claimed in claim 1, wherein the application method comprises:
applying the compound or the pharmaceutically acceptable salt thereof to prepare a protease inhibitor.
